(19) United States
(12) Patent Application Publication
(10) Pub. No.: US 2020/0357487 A1
(43) Pub. Date: Nov. 12, 2020

(54) COMPUTER-IMPLEMENTED METHOD AND SYSTEM FOR DETERMINING A DISEASE STATUS OF A SUBJECT FROM IMMUNE-RECEPTOR SEQUENCING DATA

(71) Applicant: CAMBRIDGE ENTERPRISE LIMITED, Cambridge (GB)

(72) Inventors: Elizabeth SOILLEUX, Cambridge (GB); Anna AUER-FOWLER, Liverpool (GB)

(73) Assignee: CAMBRIDGE ENTERPRISE LIMITED, Cambridge (GB)

(21) Appl. No.: 16/761,185

(22) PCT Filed: Nov. 5, 2018
(86) PCT No.: PCT/GB2018/053198

§ 371 Date: May 1, 2020

(30) Foreign Application Priority Data
Nov. 3, 2017 (GB) 1718238.7

(51) Int. Cl. G16B 40/30; G16B 20/00; G16B 50/30; G16H 50/20

(52) U.S. Cl. CPC G16B 40/30; G16H 50/20; G16B 50/30; G16B 20/00

(57) ABSTRACT

The present invention provides a method of determining the disease status in a subject, the method comprising: a) obtaining sequence data for the T-cell and/or B-cell receptor repertoire in a sample obtained from a subject; b) determining a data set of overlapping k-mer frequencies in the sequence data obtained in a); c) reducing data dimensionality of the data set of k-mer frequencies determined in b) to generate a reduced data set of k-mer frequencies; d) classifying the sample according to disease status based on the reduced data set determined in c) by performing any suitable form of statistical analysis, including, but not limited to, cluster analysis, on the reduced data set; and e) optionally applying the approach described in a) to d) to classify samples of unknown disease status on the basis of their similarity to samples of known disease status.

The present invention relates to a method for determining the disease status of a subject, and particularly although not exclusively, to determining the coeliac disease status of a subject. In particular, the invention relates to the use of k-mer analysis of the T-cell or B-cell receptor repertoire in diagnostic assays, in particular for coeliac disease. The approach is generalisable to the diagnosis or prediction of prognosis in any condition that is mediated or modulated by the immune system, including autoimmunity, hypersensitivity, allergy, transplantation, transplant rejection, cancer and all forms of neoplasia, infectious diseases and vaccination.

Coeliac disease is a T-cell mediated autoimmune disorder of the digestive system, in particular the small intestine, characterised by an adverse reaction to gluten. Classic symptoms include gastrointestinal problems such as chronic diarrhoea, abdominal distension, malabsorption, loss of appetite, and among children failure to grow normally. The prevalence of formally diagnosed coeliac disease varies between different regions of the world, from as few as 1 in 300 to as many as 1 in 40, with an average of between 1 in 100 and 1 in 170 people. In developed countries, it is estimated that 80% of cases remain undiagnosed, usually because of minimal or absent gastrointestinal complaints and poor awareness of the condition.

Whilst the avoidance of gluten can ameliorate the symptoms of coeliac disease, evidence suggests that coeliac disease is underdiagnosed or often misdiagnosed as other conditions such as irritable bowel syndrome (IBS).

Coeliac disease is caused by a reaction to gluten, a term which encompasses various proteins found in wheat and in other grains such as barley and rye. In subjects with coeliac disease an abnormal immune response is observed in response to gluten which may lead to the production of several different autoantibodies which may be used as markers of the disease. In the small bowel, gluten may cause an inflammatory reaction and may produce shortening of the villi lining the small intestine (villous atrophy), leading to malabsorption.

The only known effective treatment for coeliac disease is a strict lifelong gluten-free diet, which leads to recovery of the intestinal mucosa, improves symptoms, and reduces the risk of developing complications in most people. If untreated, coeliac disease may result in cancers, also known as neoplasia or malignancy, such as intestinal lymphoma. Other complications of coeliac disease may include nutritional deficiency, osteoporosis and neurologicalproblems.

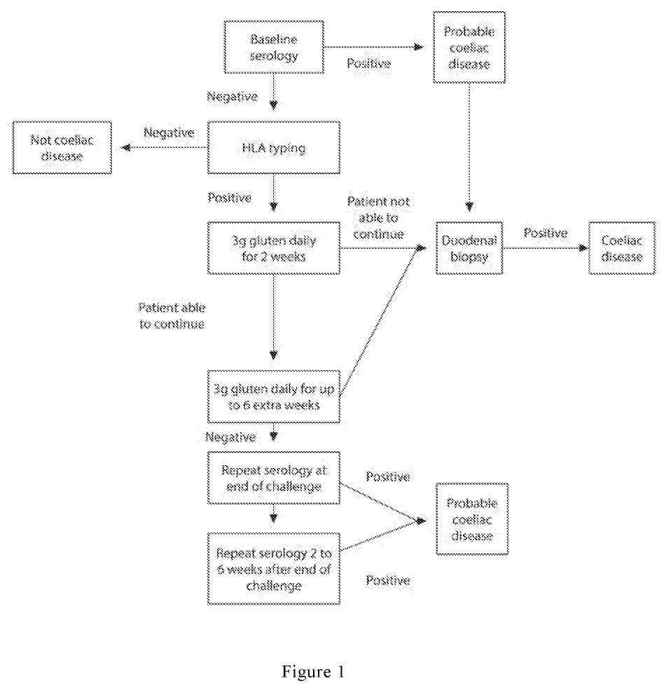

Currently diagnosis of coeliac disease is typically made by a combination of blood antibody tests and intestinal biopsy. Making the diagnosis is not always straightforward. The gold standard for diagnosis of coeliac disease is histopathological examination of small bowel biopsies, following initial serological investigations on patients in whom coeliac disease is suspected. Serological screening tests have been developed over the years and those currently in use are anti-gliadin antibodies, anti-endomysial and anti-tissue transglutaminase (TTG) antibodies with the latter two being the most accurate. Anti-endomysial tests showed a lower sensitivity than for dual (IgA and IgG) anti TTG antibodies (62-68% vs 90-92%) but a higher specificity (80-99% vs 81-83%). Combination testing of both endomysial and TTG antibodies has shown a slight increase in positive predictive value, negative predictive value and specificity, at the expense of sensitivity. However, it should be noted that, antibody-based tests for coeliac disease have, in general, been validated against the rather imperfect gold standard, histopathology, meaning that the sensitivity and specificity figures given are themselves of uncertain validity. FIG. 1 illustrates the current general approach taken to the diagnosis of coeliac disease in adults; this is taken from Schuppan at al. (2014) Gut July; 62(7):996-1004. In children the pathway to diagnosis is even more complicated, as discussed by Vriezinga, S. L. et al. (2015) Nat. Rev. Gastroenterol. Hepatol. doi:10.1038/nrgastro.2015.98.

In some centres, genetic typing of subjects at the HLA locus is also used in the diagnosis. This is because >99% of patients with coeliac disease are positive for HLA-DQ2 and/or HLA-DQ8. Therefore, patients with other HLA types and some features suspicious for coeliac disease are generally assumed either to have non-coeliac gluten-sensitivity or not have coeliac disease at all.

Unfortunately current diagnostic tests are far from perfect, and it is not uncommon for subjects with severe symptoms to be negative for autoantibodies in the blood and to have only minor intestinal changes with normal villi. Furthermore, for the tests to be most efficacious the biopsy should be taken where the subject is on a gluten containing diet, which may mean the subject has severe symptoms, so it can be an unpleasant test. The results may still be inconclusive as the morphological changes may be mild, or the subject may not have been exposed to sufficient gluten. People may have severe symptoms and be investigated for years before a diagnosis is achieved.

The present invention has a number of advantages over the current diagnostic protocol, these advantages may include one or more of the following:
increase the predictive value of a biopsy when used in addition to a biopsy;
it may reduce or avoid the subjectivity of the analysis of a biopsy;
it may reduce or avoid the need for the subject to be exposed to gluten prior to the test; and/or
it may avoid the need for an endoscopy as the test may be carried out on a blood sample, and, if duodenal lymphocytes are needed, they are very superficial and may be obtained by less invasive means than endoscopy.

According to a first aspect the present invention provides a method of determining the disease status in a subject, the method comprising:
a) obtaining sequence data for the T-cell and/or B-cell receptor repertoire in a sample obtained from a subject;
b) determining a data set of overlapping k-mer frequencies in the T-cell and/or B-cell sequence data obtained in a);
c) reducing data dimensionality of the data set of k-mer frequencies determined in b) to generate a reduced data set of k-mer frequencies; and
d) classifying the sample according to disease status based on the reduced data set determined in c) by performing cluster analysis on the reduced data set;
e) optionally applying the approach described in a) to d) to classify samples of unknown disease status on the basis of their similarity to samples of known disease status.

In step a) the sequence data may be obtained by sequencing the T-cell and/or B-cell receptor repertoire in the sample, or being provided with the sequence data.

In an embodiment of the method of the invention the method may be used to determine the coeliac disease status of a subject. The phrase “coeliac disease status” includes any distinguishable manifestation of coeliac disease. In particular the method of the invention may allow coeliac disease to be distinguished from other conditions such as non-coeliac gluten-sensitive enteropathy, irritable bowel syndrome or wheat allergy.

The method of the invention may also or alternatively be used to diagnose gluten sensitive disorders to be distinguished and diagnosed. The method of the invention may allow subjects with a gluten sensitivity to be identified.

In another embodiment the method of the may be used to determine the status of any condition or disease mediated of modulated by the immune system in a subject. The disease or condition may be selected from the group comprising an autoimmune condition, hypersensitivity, allergy, transplantation, transplant rejection, cancer, all forms of neoplasia, infectious diseases, and vaccination.

In another embodiment the method of the invention may be used to provide a prognosis for a subject with a tumour on the basis of their tumour infiltrating lymphocyte population or related lymphocyte populations in peripheral blood.

In a further embodiment the method of the invention may be used to diagnose cancer or neoplasia, or to provide a prognosis for a subject with cancer or any type of neoplasia, neoplasia encompassing both benign and malignant tumours. The cancer or neoplasm may be a carcinoma, B-cell lymphoma, leukaemia, mesothelioma, germ cell tumour, sarcoma, melanoma or adenoma. In another embodiment the method of the invention may be used to predict the likelihood of transplant rejection and/or graft-versus-host disease. A further embodiment may permit the assessment of target or severity of an allergic response and/or the individual's prognosis with respect to the allergic response, either with or without immunomodulatory therapy.

In another embodiment the method of the invention may be used to diagnose or provide a prognosis for an immune-mediated disease, such as an autoimmune disease, such as multiple sclerosis (CSF T-cell and/or B-cell receptor repertoire), pre-type I insulin-dependent diabetes mellitus (for example, blood T-cell and/or B-cell receptor repertoire), polymyositis, dermatomyositis (for example, blood, skin or muscle biopsy T-cell and/or B-cell receptor repertoire), systemic lupus erythemtososus (SLE) (T-cell and/or B-cell receptor repertoire from multiple organs and/or samples, including serous fluid), rheumatoid arthritis (T-cell and/or B-cell receptor repertoire from joint aspirate fluid), HLA-B27-associated arthritides (e.g., ankylosing spondylitis), autoimmune hepatitis, primary biliary cirrhosis or primary sclerosing cholangitis. In another embodiment the method of the invention may be used to diagnose an inflammatory disease. The inflammatory disease may be inflammatory bowel disease, or an inflammatory skin disease, such as eczema, psoriasis, pityriasis, systemic lupus erythematosus or lichen planus. The method may be able to distinguish ulcerative colitis from Crohn's disease and/or from unaffected. In another embodiment the method of the invention may be used to diagnose vasculitis, or provide a prognosis for a subject with vasculitis.

The method may also be able to predict the response of a subject to a particular therapy for the treatment of tuberculosis and mycobacterial infection (e.g., leprosy). The method may also be able to provide a prognosis for subjects with viral, bacterial, fungal or parasitic infections, including, but not limited to EBV, HIV or another viral infection, or with Lyme disease, mycobacterial infection (e.g., tuberculosis), Leishmaniasis or dengue. The method may be able to predict outcome of vaccination (e.g., against hepatitis B or melanoma), for example whether the patient has developed a likely protective response against the infection or tumour, respectively. Similarly, the method may be able to determine the severity or likely outcome of transplant rejection.

The sample may be a bodily fluid, such as blood or a product derived from blood, or lymph or a product derived from lymph. Bodily fluids might also include pericardial, pleural or ascitic (peritoneal) fluid, joint aspirate fluid or urine. Alternatively, the sample may be a biopsy sample, for example a duodenal sample, or a fine needle aspirate sample, such as a lymph node sample. The sample might also be a buccal scrape or skin scrape. Preferably, the method does not include the step of obtaining the sample from a subject.

In step a) the sequence data of the T-cell and/or B-cell receptor repertoire may be determined by sequencing the T-cell and/or B-cell receptor repertoire. The T-cell and/or B-cell receptor repertoire refers to substantially all the different T-cell and/or B-cell receptors in a subject, or a sample obtained therefrom. When determining the T cell and/or B-cell repertoire, the variable region, the VDJ region, of the receptor may be analysed by sequencing, typically next generation sequencing. When determining the T-cell receptor repertoire and/or B-cell receptor repertoire, the CDR3 region of the T-cell receptor and/or B-cell receptor may be analysed by sequencing, typically next generation sequencing. When determining the T-cell and/or B-cell receptor repertoire the sequence of one or more of the following genes may be determined: TRA which encodes TCRa, TRB which encodes TCRO, TRG which encodes TCRy and TRD which encodes TCRS and/or IGH, which encodes B-cell receptor/immunoglobulin heavy chain, IGK, which encodes B-cell receptor/immunoglobulin kappa light chain and IGL, which encodes B-cell receptor/immunoglobulin lambda light chain. The variable region, specifically the CDR3 region within it, undergoes complex rearrangement at a genomic level during T-cell and B-cell development. Particular T-cell and B-cell receptors, which are able to bind different antigens, are likely to be selected for during immune responses, thus increasing their relative frequencies, in different disease or other physiological or pathophysiological states. By using next generation sequencing the sequence of one or more of TRA, TRB, TRG, TRD, IGH, IGK and IGL may be readily determined. The DNA or RNA sequence may be determined. If the DNA sequence is used, all sequences which contain stop codons or are out of frame may be discarded.

In step a) the step of obtaining sequence data may comprises the step of: sequencing the T-cell and/or B-cell receptor repertoire in the sample; and/or being provided with the sequence data; or
sequencing the T-cell receptor repertoire in the sample at DNA level; and/or being provided with the DNA sequence data; or
sequencing the T-cell receptor repertoire in the sample at RNA level; and/or being provided with the RNA sequence data; or
sequencing the T-cell and receptor repertoire in the sample at amino acid level; and/or being provided with the amino acid sequence data; or
sequencing the B-cell receptor repertoire in the sample at DNA level; and/or being provided with the DNA sequence data; or
sequencing the B-cell receptor repertoire in the sample at RNA level; and/or being provided with the RNA sequence data; or
sequencing the B-cell receptor repertoire in the sample at amino acid level; and/or being provided with the amino acid sequence data.

When analysing the T-cell receptor repertoire for TRA and TRB, in the case of coeliac disease, for example, it may be necessary to consider whether the HLA type is (i) HLA-DQ2, (ii) HLA-DQ8, (iii) both HLA-DQ2 and HLA-DQ8 or (iv) neither HLA-DQ2 nor HLA-DQ8 and separate reference databases for clustering of test samples may be required for each of these groups. Similarly, for other conditions, it may be necessary to use more refined HLA typing, and to analyse samples of certain HLA types together.

The number of individual sequencing reads obtained from a sample may be around 100,000 sequences for the repertoire of a particular T-cell or B-cell locus (e.g., TRB, TRG, IGH) in any given patient sample, but a total number of reads of 50,000 per sample is typically sufficient to allow analysis in the embodiments described. It can be appreciated that utilising a higher sequencing depth is generally preferable.

In the sequencing step a percentage of the T-cell receptor or B-cell receptor repertoire may be amplified. This means that of the total number of potential unique T-cell receptor or B-cell receptor sequences present in the sample, only a certain percentage will be amplified and sequenced and this percentage may vary between samples. This percentage may be at least 0.1%, at least 1%, at least 5%, at least 10%, at least 20%, at least 30%, at least 40% or at least 50%.

In an embodiment at least the sequences of one of the T-cell receptor or B-cell receptor loci, that is one of TRA, TRB, TRG, TRD, IGH, IGK and IGL, are used to determine the coeliac disease status or other disease status or other physiological or pathophysiological status of a subject. For example, in an embodiment, the TRG repertoire of a particular sample, e.g., a biopsy or a blood sample, from an individual may contain at least 10 sequences, at least 100 sequences, at least 1,000 sequences, or at least 10,000 sequences or at least 20,000 sequences.

The sequence data of step a) may be a library of sequences representing the T-cell and/or B-cell receptor repertoire in the sample provided by the subject. The library may contain the DNA/RNA sequence, and/or it may contain the protein sequence derived from the DNA/RNA. Preferably the output is a library of protein sequences representing the T-cell and/or B-cell receptor repertoire in the sample.

The data set may be obtained by analysing the sequence data obtained in a) to identify the frequency of occurrence of k-mers of a specific length. For example, if a k-mer of 4 is used, the analysis would determine the frequency of occurrence of all possible combinations of 4 consecutive nucleotides in a particular DNA/RNA sequence or all possible combinations of 4 consecutive amino acids in a particular protein sequence, similarly for a k-mer of 5 the analysis would look at the frequency of occurrence of all possible combinations of 5 nucleotides in a particular DNA/RNA sequence or all possible combinations of 5 amino acids in a particular protein sequence. When considering sequences in terms of k-mers in this methodology, the k-mers overlap, as the k-mer identification process moves along 1 nucleotide or amino acid at a time. Essentially the k-mer frequency refers to the relative or absolute frequency of occurrence of all possible substrings of length k contained in a particular string—that is the frequency of all subsequences of length k in a particular DNA/RNA or protein sequence. In general, the relative frequency is preferred and is determined using normalisation methods during the analysis. Preferably in the method of the invention k-mers of between 3 and 10 amino acids are used, more preferably between 4 and 7 amino acids. In an embodiment a k-mer of 5 amino acids is used. In a further embodiment, the k-mer is annotated with its position within CDR3, e.g. beginning or end, or beginning, middle or end. Under these conditions, k-mers with an identical sequence occurring at different points within the CDR3 sequence (e.g., at the beginning and the end) will not be considered to be equivalent during the analysis.

The data set described in b) may provide the relative or absolute frequency of each k-mer in a particular sample. It may also be considered to be a set of all k-mers contained in the T-cell and/or B-cell receptor repertoire for the sample or individual. By analysing the CDR3 amino acid sequence in this manner, the analysis may not be affected by differing absolute chain length and may only consider the occurrence of short consensus sequences, which may be at different positions. Given that the absolute sequences will likely contain short consensus sequences, by analysing the frequency of unique substrings having a defined k-mer length, the absolute sequence length is removed as a variable.

In a particular example, the number of unique CDR3s per sample are analysed and typically between 7,500 and 50,000 unique CDR3s are identified per individual. An approximate median value is 23,000, with a lower quartile value of 17,000.

Taking, for example, a k-mer length of k=5, the number of unique k-mer (5-mer) sequences for all samples can be identified. For example, in a formalin fixed paraffin embedded duodenal biopsy from a patient with coeliac disease, approximately 320,000 unique TRG k-mers of length 5 (or 5-mers) were identified. It can be appreciated that the number of total unique 5-mers in a particular data set is dependent on the number of samples measured, as well as on the number of unique CDR3 sequences and the length of each CDR3 sequence. However, if the number of samples is large, adding each additional sample generates a diminishing increase in the number of unique k-mers.

The data set may be a series or set of data indicating the absolute frequency of each k-mer in a particular sample, i.e. for a particular individual. The data set may have K elements containing a series of substrings of length k.

For a set of S samples a frequency matrix describing the relative number of k-mers observed in the sample may then be calculated. The frequency matrix scales the number of times a k-mer is observed in a sample by the total number of k-mers observed in the sample for each k-mer and for each sample, effectively giving relative k-mer frequency.

For a single sample (such as a test sample from a new patient), a frequency matrix may still be calculated utilising reference data from a reference set of samples. In this manner, the k-mer frequencies of the single test sample could be compared with the reference data set to obtain the frequency matrix.

Accordingly, it may be considered that the frequency of a k-mer in a sample is given by the number of times the k-mer is observed in the sample, scaled by the total number of k-mers observed in the sample. The frequency of each k-mer in each sample is then contained in the frequency matrix.

In a method of the invention the data set may provide a frequency of each k-mer in particular sample wherein the date set provides an absolute or a relative frequency of each k-mer in a particular sample.

Step b) may be further divided into 3 steps:
i) identifying k-mers present within the sequence data, each k-mer representing a nucleotide or amino acid combination of a specific length;
ii) determining k-mer frequencies for every k-mer identified in step i), said k-mer frequencies indicating the number of times the k-mer is present within the sequence data;
iii) scaling the k-mer frequencies by the total number of k-mers identified in the sequence data (nucleotide or amino acid sequences) in order to give relative k-mer frequencies within the sample.

Step i) may further comprise the steps of analysing the nucleotide or amino acid sequences, usually against samples for which diagnosis or prognosis is known (the “ground truth” or training set) to determine an optimum k-mer substring length. For example, in step b) if a k-mer of 4 is used, all possible combinations of 4 nucleotides either present or potentially present in a particular DNA/RNA sequence or all combinations either present or potentially present of 4 amino acids within a particular protein sequence are identified. It may be appreciated that, where the combination of nucleotides actually present is analysed, then a further step of identifying the combination of nucleotides present in the DNA/RNA sequence may be undertaken. Similarly, for a k-mer of 5 the analysis would look at the frequency of occurrence of all possible combinations of 5 nucleotides in a particular DNA/RNA sequence or all possible combinations of 5 amino acids in a particular protein sequence.

The output of steps i) and ii) may be a data set providing the frequency of each k-mer in a particular sample. It may also be considered to be a set of all k-mers contained in the T-cell and/or B-cell receptor repertoire for the sample or individual. By analysing the CDR3 amino acid sequence in this manner, the data are standardised to account for differing absolute chain length, and for consensus motifs that occur in different CDR3 sequences at different positions within the sequences.

In particular, once the set of all k-mers contained within the repertoires have been identified, a k-mer frequency may then be determined based on the total number of times that the k-mer is observed in the sample. Using the k-mer analysis described above, all k-mers in the T-cell and/or B-cell receptor repertoire of sample s, for s=1, . . . , S can be identified and denoted xₛ, and then a set K of all k-mers contained within x₁, . . . xₛ, containing K elements is denoted by K={k₁, . . . , k_{K}} can also be computed.

In order to normalize the k-mer frequency data, the number of times that the k-mer is observed in the sample may be scaled (in step iii) by the total number of k-mers observed in that sample. This accounts for different sequencing depths per sample.

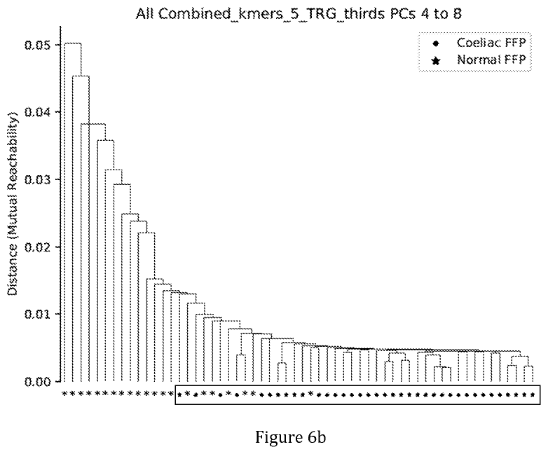

In an extension of the k-mer approach, individual k-mers may be annotated by their position within the CDR3 sequence, for example whether the amino acids making up the k-mer occur (a) at the beginning or the end of the CDR3 amino acid sequence or whether they occur (b) at the beginning, in the middle or at the end of the CDR3 amino acid sequence or (c) at any specifically defined position within the CDR3 sequence (and the ability of k-mer annotation by position within CDR3 is shown in FIG. 6). By extension, other annotations of k-mers are also possible, for example whether they occur in the context of a particular CDR1 and/or CDR2 sequence or, in the case TCR gamma, in the context of a particular of CDR1, CDR2 and/or CDR4/HV4 sequence. k-mers may also be annotated as to whether they occur in the context of usage of a particular V segment, D segment and/or J segment. In the case of TCR beta, TCR gamma and Ig heavy chain, k-mers may also be annotated as to whether they occur in the context of usage of a particular V segment, D segment, J segment or C segment.

The method of the invention may further include providing a data set which further provides information on relative positions of k-mers of amino acids or bases within the T-cell receptor or B-cell receptor amino acid or base sequence, optionally within the CDR3 amino acid or base sequence. For example their position at the beginning, in the middle or at the end of the CDR3 sequence or their position at the beginning or the end of the CDR3 sequence, or their position with the CDR3 sequence defined in any other way. In the situation, for example a particular k-mer sequence occurring at the start of the CDR3 sequence would be considered not to be identical to the same sequence occurring at the end of the CDR3 sequence.

In an alternative embodiment, the data set may further provide information on relative associations of k-mers with a particular CDR1, CDR2 or TRG CDR4/HV4 sequence. In this situation a particular Kmer sequence occurring in conjunction with a particular CDR1 sequences would be considered not to be identical to the same sequence occurring with a different CDR1 sequence. In this situation a particular k-mer sequence occurring in conjunction with a particular CDR2 sequences would be considered not to be identical to the same sequence occurring with a different CDR2 sequence. In this situation a particular k-mer sequence occurring in conjunction with a particular CDR4/HV4 sequences would be considered not to be identical to the same sequence occurring with a different CDR4/HV4 sequence. The skilled person will appreciate that k-mer association with any combination of two or more of CDR1, CDR2 or TRG CDR4/HV4 sequence may also be considered in this way.

In an embodiment, this normalization is the calculation of a frequency matrix describing the relative total number of k-mers observed in the sample. The frequency matrix scales the number of times a k-mer is observed in a sample by the total number of k-mers observed in the sample for each k-mer and for each sample.

The frequency matrix, a S×K k-mer frequency matrix, M, for subject indexed i, and k-mer indexed j, Mij is the number of times k-mer j appears in xi, scaled by the total number of k-mers in xi and may be calculated by:

$${M}_{i , j} = \frac{{\sum }_{l} {I}_{{x}_{il} = {k}_{j}}}{{\sum }_{l} {\sum }_{m} {I}_{xil} = {k}_{rx}}$$

where Ixil=kj is an indicator function which takes the value 1 when xil=kj and 0 otherwise (where xil is the lth k-mer in sample i and kj is the jth k-mer in the set K, as defined above).

In step c) dimensionality reduction to reduce the data complexity is performed on the k-mer frequencies from step b). An example of this is principal component analysis (PCA); a mathematical transformation that reduces the dimensions of the data whilst capturing the major sources of variation. Other data analysis approaches that can reduce the numbers of dimensions, such as contrastive principal component analysis (cPCA) or variable selection (also known as feature selection), are also applicable. Methods that can provide either dimensionality reduction, subsequent classification or both include linear discriminant analysis, generalised discriminant analysis, quadratic discriminant analysis and canonical correlation analysis. Methods that can provide classification, usually following a dimensionality reduction method, include clustering approaches (which may or may not be hierarchical and which can be supervised or unsupervised), support vector machines, logistic regression, nearest neighbour analysis, decision trees and neural network-based approaches. Details of such methodologies may be found in standard mathematical textbooks (Hastie, T; Tibshirani, R; Friedman, J. The elements of statistical learning: Data Mining, Inference, & Prediction. 2nd Edition. Springer, N Y 2009).

Where the k-mer frequencies are represented as a frequency matrix, step c) may comprise the steps of determining eigenvectors of the covariance matrix of M. In an embodiment the step of determining eigenvectors includes the step of normalising the matrix.

In an embodiment, the step of analysing by principal component analysis includes the step of obtaining a dot product of the frequency matrix for a sample and the eigenvectors of the covariance matrix of M.

Principal component analysis in the manner described above allows the multi-dimensional and highly correlated data contained within the frequency matrix to be transformed onto a relative coordinate system. An advantage of principal component analysis in the manner described is that the principal components (i.e. the relative variance in the data containing the highest variance) can be determined. Data in minor principal components (i.e. having lower variance) can be discarded.

As described above, the output from step c) is an indication of which k-mer frequencies have the greatest relative variance for each sample.

Step c) may also include a further step iv) of performing principal component analysis to calculate the principal components of the scaled k-mer frequencies.

As noted in step iv), the principal components of the data may then be determined. This transforms the data onto a new coordinate system, with the first principal component containing the greatest data variance. Principal component analysis is a mathematical transformation that reduces the dimensions of the data whilst capturing the major sources of variation.

Where the data set of k-mer frequencies are a frequency matrix, step c) may comprise the steps of determining eigenvectors of the covariance matrix of M. In an embodiment the step of determining eigenvectors includes the step of normalising the matrix.

In embodiments, the step of analysing by principal component analysis may comprise the step of obtaining a dot product of the frequency matrix for a sample and the eigenvectors of the covariance matrix of M.

Principal component analysis in the manner described above allows the multi-dimensional and highly correlated data contained within the frequency matrix to be transformed onto a relative coordinate system. An advantage of principal component analysis in the manner described is that the principal components (i.e. the relative variance in the data containing the highest variance) can be determined. Data in minor principal components (i.e. having lower variance) can be discarded.

In a particular example, for the frequency matrix M, if wᵢ=wᵢ₁, . . . , w_{i, K} is the i^{th} eigenvector, then for samples, the i^{th} principal component is given by Mₛ·w_{b} where Mₛ is the vector obtained from the s^{th} row of M.

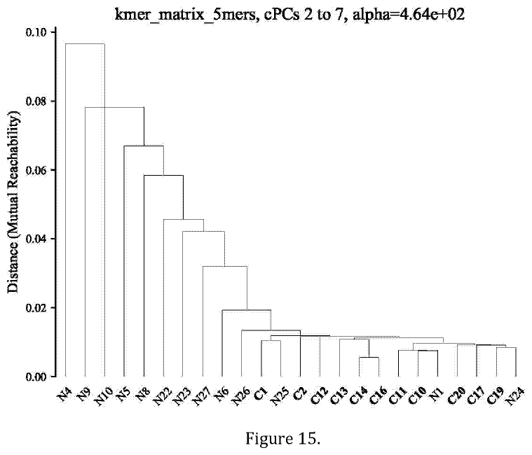

Other methods of dimensionality reduction could be used instead of PCA, for example contrastive PCA (FIG. 15) or variable (or feature) selection. The reduced data set obtained using any alternative method would then be considered an output from step c) and therefore an input for step d).

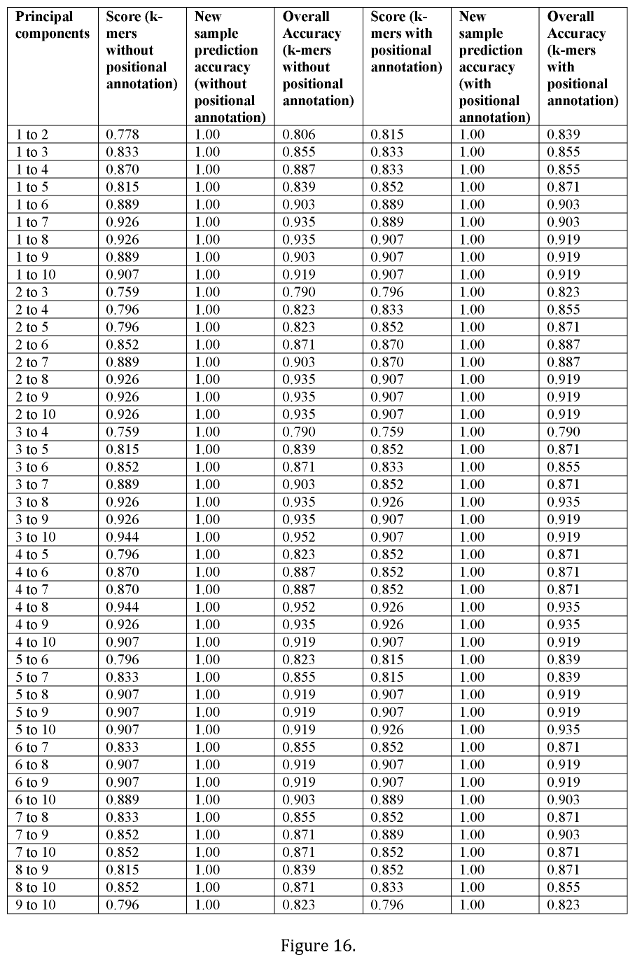

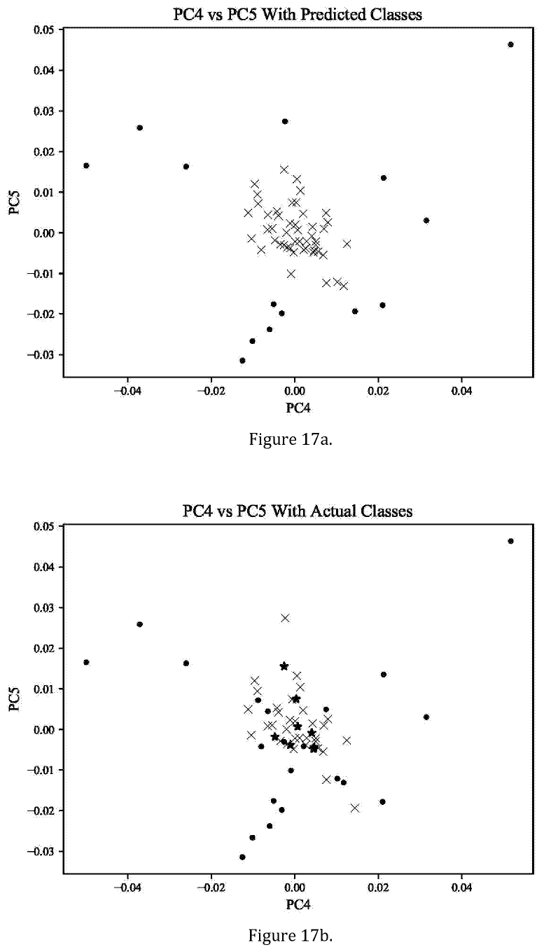

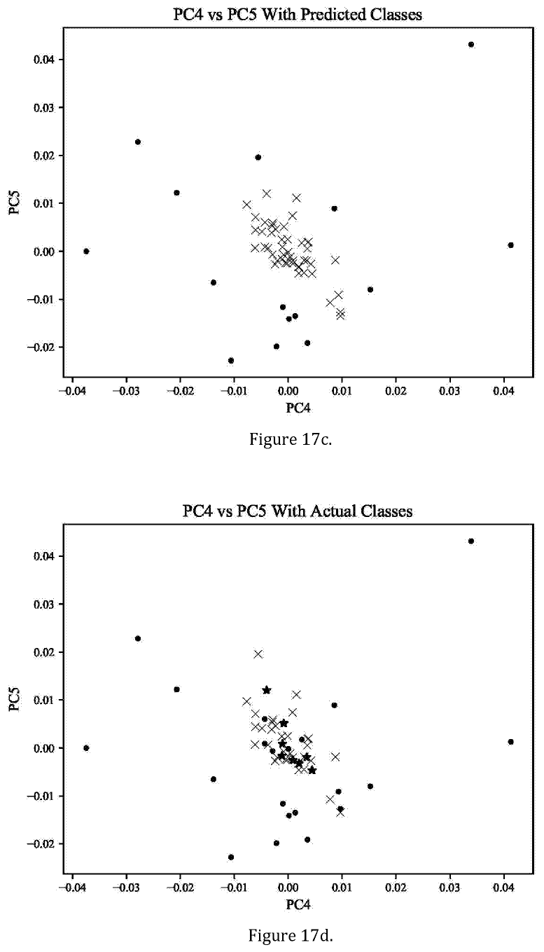

In step d) the data from step c) is classified according to disease status or prognosis, for example using clustering, which could include hierarchical clustering and which may be performed in a supervised or unsupervised manner. Parameters (as described below) can be chosen to optimise the separation between cases and controls, and then applied to new, test samples in order to determine their disease status. When using clustering, the result is that the coeliac samples (or any other sample type with a particular diagnosis, prognosis or disease status of interest) form a single cluster with high levels of similarity whilst the healthy samples form several smaller clusters with lower levels of similarity. Other classification methods, such as quadratic discriminant analysis (FIGS. 16 and 17), are able to distinguish between cases and controls, identifying cases as a highly similar, close group whilst controls are more diverse and widely separated. This indicates that the T-cell receptor repertoires of coeliac samples exhibit similar k-mer patterns, whilst healthy samples are more diverse. Analogous results may be seen in a wide range of other immunologically mediated or immunologically modulated conditions.

The clustering may be undertaken using the principal components indexed according to a parameter p, where p is the principal component, and p=2, 3, 4. Ward's minimum variance method may be used for the clustering.

From this analysis, samples may be indexed, meaning that they are grouped or clustered based on their relative variance or principal components. Samples having the smallest increased variance can then be merged. The parameters used can be optimised using a reference panel in order to obtain the best separation between patients in different classifications, and then applied to new test samples.

In some embodiments, the first principal component may be discarded. Typically the first principal component describes batch variability between samples. Accordingly, the principal components 2, to 10 may generally be used for indexing, either as individual principal components or combinations of two or more principal components.

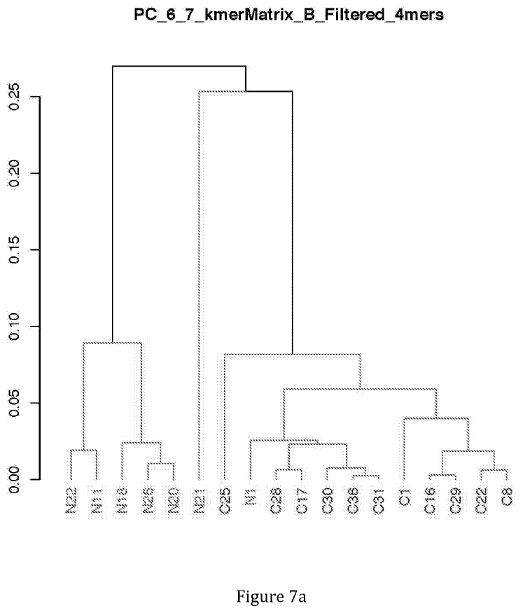

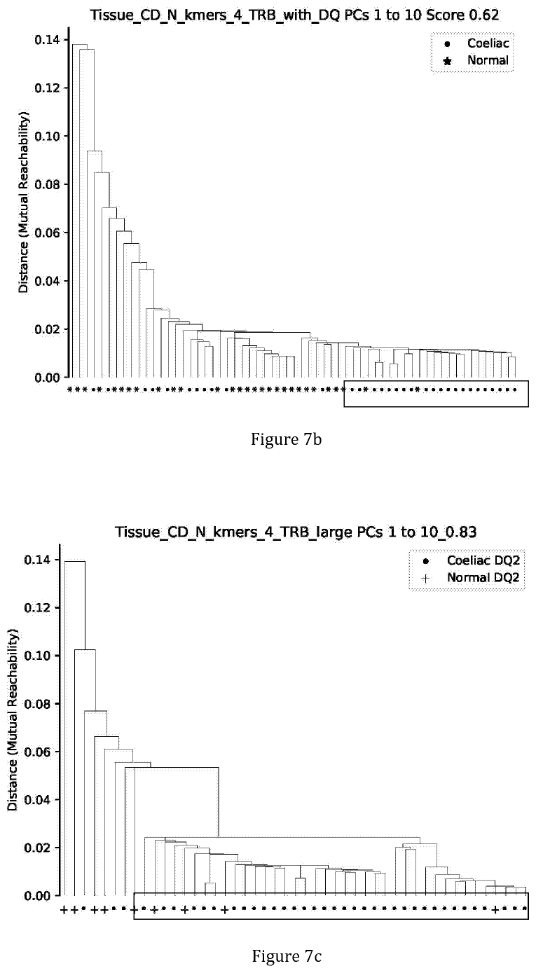

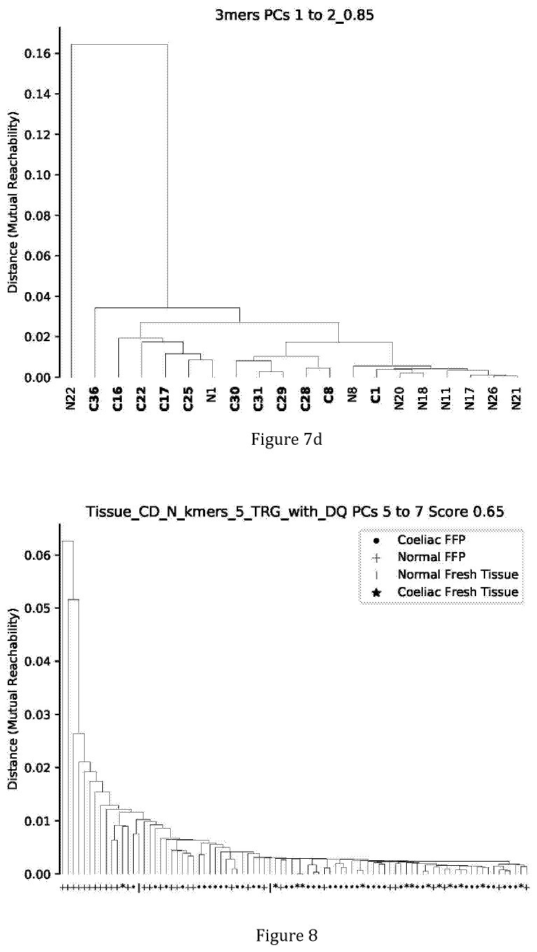

Other methods of classification may be used instead of or clustering, for example quadratic discriminant analysis, neural networks or other forms of clustering. The skilled person will also appreciate that k-mer analysis outputs from several loci, e.g., TCR beta and TCR gamma (and/or any other combination of two or more of Ig heavy chain, Ig kappa light chain, Ig lambda light chain, TCR alpha, TCR beta, TCR gamma and TCR delta) may be combined using these methodologies (FIG. 7d), in order to separate individuals with different disease and/or prognostic status.

The step of classifying the sample further might include the step of comparing an outcome of the k-mer analysis and/or subsequent classification with reference samples or data in a database containing reference k-mer data and/or subsequent classification data from reference coeliac samples and/or reference non-coeliac samples.

The step of classifying the sample further might include the step of comparing an outcome of the k-mer analysis and/or subsequent classification with reference samples or data in a database containing reference k-mer data and/or subsequent classification data from reference disease samples and/or reference normal samples.

The step of classifying the sample further might include the step of comparing an outcome of the k-mer analysis and/or subsequent classification with reference samples or data in a database containing reference k-mer data and/or subsequent classification data from reference samples from one disease and/or reference samples from another disease.

The step of classifying the sample further might include the step of comparing an outcome of the k-mer analysis and/or subsequent classification with reference samples or data in a database containing reference k-mer data and/or subsequent classification data from reference samples with three or more known different disease statuses.

The step of classifying the sample further might include the step of comparing an outcome of the k-mer analysis and/or subsequent classification with reference samples or data in a database containing reference k-mer data and/or subsequent classification data from reference samples with known physiological or pathophysiological statuses.

The output of step d) is typically either a numerical assessment, such as a ratio, percentage or relative likelihood of a particular prognostic status or disease status, for example gluten sensitivity, based on cluster position; or a yes/no, again based on cluster position, but with defined cut-offs. Preferably the output is a yes/no based on cluster position, with a sensitivity/specificity. Allowing a user to define cut-offs would allow them to have higher/lower sensitivity/specificity as desired. This is broadly equivalent to saying yes with x % certainty or no with y % certainty.

In order to do this, a database of the T-cell and/or B-cell receptor repertoires of samples (derived from duodenum or blood or other sites) from patients with a particular prognostic status or disease status, for example coeliac disease and those who do not have the condition are utilised and the analysis described above run, ideally in a fully automated form, comparing each test sample of unknown disease status with the samples in the database, to determine which it clustered with.

Accordingly, using the described method, k-mer data generated from samples from patients with unknown disease status can then be compared to data obtained from normal or reference cases, with known disease status, or annotated data sets, with known disease status, from a reference database. This could either give a ratio, percentage or relative likelihood of coeliac disease, or other disease status, or a yes/no answer, depending on how clear the likelihood is. It can be appreciated that there will always actually be a ratio, percentage or relative likelihood, but, if there is clear separation between coeliac disease, or other disease status, and normal, then a definite cut-off could be determined. However, a spectrum is typically present.

Accordingly, using this analysis of sequence data from the T-cell and/or B-cell receptor repertoire in a sample obtained from a subject allows diagnosis of coeliac disease/gluten sensitivity, or a wide range of other immunologically modulated conditions to be diagnosed or prognosis to be predicted.

The method of the invention allows subjects with coeliac disease and/or gluten sensitivity to be separated from those who do not have coeliac disease and/or gluten sensitivity.

The method of the invention has the advantage that it can be used to detect coeliac disease in a subject even when the subject is on a gluten free diet. The sensitivity of the method of the invention allows immune cells responsible for the disease to be detected at levels lower than the current tests can detect. The test may be carried out on samples obtained from subjects who have a gluten free diet.

The method of the invention may be used to diagnose whether or not a subject has coeliac disease, or to provide a prognosis for a subject with coeliac disease.

The method of the invention may be used to diagnose Crohn's disease, by comparing data from a test sample of unknown disease status with data from known samples from Crohn's disease and normal.

The method of the invention may be used in the diagnosis of ulcerative colitis, by comparing data from a test sample of unknown disease status with data from known samples from ulcerative colitis and normal.

The method of the invention may be used in distinguishing between Crohn's disease and ulcerative colitis, optionally in order to avoid a diagnosis such as indeterminate colitis, by comparing data from a test sample of unknown disease status with data from known samples from Crohn's disease and ulcerative colitis.

The method of the invention may be used for use in the determination of prognosis in melanoma patients, by comparing data from a test sample from a melanoma patient of unknown prognosis or outcome with data from melanoma patient samples with known prognosis or outcome.

The method of the invention may be used for use in the diagnosis of autoimmune conditions, including but not limited to such as multiple sclerosis, pre- or early type I insulin-dependent diabetes mellitus, polymyositis, dermatomyositis, systemic lupus erythemtososus (SLE), rheumatoid arthritis, HLA-B27-associated arthritides (e.g., ankylosing spondylitis), autoimmune hepatitis, primary biliary cirrhosis and primary sclerosing cholangitis, by comparing data from a test sample of unknown disease status with data from known samples from one or more known autoimmune condition(s) with or without normal samples.

The method of the invention may be used in the prediction of prognosis of autoimmune conditions (including but not limited to such as multiple sclerosis, pre- or early type I insulin-dependent diabetes mellitus, polymyositis, dermatomyositis, systemic lupus erythemtososus (SLE), rheumatoid arthritis, HLA-B27-associated arthritides (e.g., ankylosing spondylitis), autoimmune hepatitis, primary biliary cirrhosis and primary sclerosing cholangitis), by comparing data from a test sample of unknown disease status with data from samples with known severity or outcome in autoimmune conditions.

The method of the invention may be used in the diagnosis of hypersensitivity conditions, by comparing data from a test sample of unknown disease status with data from known samples from a known hypersensitivity condition and normal or any other suitable comparator condition or physiological/pathophysiological status.

The method of the invention may be used in the prediction of prognosis of hypersensitivity conditions, by comparing data from a test sample of unknown prognosis in a hypersensitivity condition with data from samples in a known hypersensitivity condition with unknown severity, outcome status or precipitating antigen with data from hypersensitivity condition samples with known severity, outcome status or precipitating antigen.

The method of the invention may be used in the diagnosis of allergic conditions, by comparing data from a test sample of unknown disease status with data from known samples from a known allergic condition and normal or any other suitable comparator condition and/or physiological/pathophysiologicalstatus.

The method of the invention may be used in the prediction of prognosis of allergic conditions, by comparing data from a test sample of unknown prognosis in a hypersensitivity condition with data from samples in a known allergic condition with unknown severity, outcome status or precipitating antigen with data from allergic condition samples with known severity, outcome status or precipitating antigen.

The method of the invention may be used in the diagnosis of transplant rejection of any organ or tissue, by comparing data from a test sample of unknown disease status with data from known samples with a known rejection status and normal or any other suitable comparator condition or physiological/pathophysiological status.

The method of the invention may be used in the prediction of prognosis or outcome in transplant rejection of any organ or tissue, by comparing data from a test sample of unknown disease status with data from samples with a known transplant rejection status, prognosis or outcome.

The method of the invention may be used in the prediction of prognosis or outcome in cancer or any form of neoplasia, by comparing data from a test sample of unknown cancer or neoplasia outcome status with data from known cancer or neoplasia samples with a known prognosis or outcome status.

The method of the invention may be used in the diagnosis of an infectious disease, by comparing data from a test sample of unknown infectious disease status with data from samples from individuals with a particular infectious disease status and normal and/or other infectious diseases.

The method of the invention may be used in the prediction of prognosis or outcome of an infectious disease (of any organ or tissue), by comparing data from a test sample of unknown infectious disease prognosis or outcome status with data from samples from individuals with that particular infectious disease with known prognosis or outcome status.

The method of the invention may be used in the prediction of prognosis or outcome of a vaccination, by comparing data from a test sample of unknown vaccination prognosis or outcome status with data from samples from individuals with particular prognosis or outcome statuses following vaccination.

The method of the invention may be used in the determination of the specific response to one or more antigens following vaccination, by comparing data from a test sample of unknown vaccination response with data from samples from individuals with a known specific immune response to one or more specific antigens.

The method of the invention may be used in the determination of the specific response to one or more antigens in the setting of infection, by comparing data from a sample of unknown infection status with data from samples from individuals with a known specific immune response to one or more specific antigens.

The method of the invention may be used in the diagnosis or prediction of prognosis or outcome status of any particular physiological or pathophysiological state that is, at least in part, determined, mediated by or modulated by the immune system, by comparing data from a test sample of unknown diagnosis, prognosis or outcome status with data from samples with a known diagnosis, prognosis or outcome status.

The method of the invention may be used in the determination of biological similarity with respect to the immune system of any group(s) of samples for the purpose of diagnosis.

The method of the invention may be used in the determination of biological similarity with respect to the immune system of any group(s) of samples for the purpose of prediction of prognosis or outcome.

The method of the invention may be used in the determination of physiological or pathophysiological state with respect to the immune system of any group(s) of samples for any purpose that involves comparison of the samples with samples from subjects with known physiological or pathophysiological states.

The method of the invention may be used in the determination of biological similarity with respect to the immune system of any group(s) of samples for any purpose that involves comparison of the similarity or differences between the samples.

The method of the invention may be used in the determination of whether there is a specific response to one or more antigens in a sample, by comparing data from that sample with data from samples from subjects with a known specific immune response to that or those specific antigen(s).

The method of the invention may be used with a sample obtained from humanised or non-humanised rodents and from other species, including, but not limited to, monkeys, apes, cats, dogs, cows, horses, rabbits or rodents.

The method of the invention may be used in the diagnosis and/or prediction of prognosis in any animal with T-cell receptors and/or B-cell receptors that undergo genomic rearrangement, including any jawed vertebrate from fishes to mammals, for example humans, monkeys, apes, cats, dogs, cows, horses, rabbits, rodents chickens and zebra fish.

The sequence data used in the k-mer analysis may comprise sequences of any one or two or more of the following genes: TRA, TRB, TRG and TRD (considered components of the T-cell receptor repertoire) IGH, IGK and IGL (considered components of the B-cell receptor repertoire).

The sequence data used in the k-mer analysis may comprise one or more of sequences of TRA; sequences of TRB; sequences of TRG; sequences of TRD; sequences of IGH; sequences of IGK; sequences of IGL; and sequences of TRA and TRB, TRG and/or TRD.

The sequence data used in the k-mer analysis may comprise sequences of TRA and TRB, combined with a 1:1 weighting or any other weighting between 10,000:1 and 1:10,000.

The sequence data used in the k-mer analysis may comprise sequences of TRG and TRD, combined with a 1:1 weighting or any other weighting between 10,000:1 and 1:10,000.

The sequence data used in the k-mer analysis may comprise sequences of TRA and TRG, combined with a 1:1 weighting or any other weighting between 10,000:1 and 1:10,000.

The sequence data used in the k-mer analysis may comprise sequences of TRA and TRD, combined with a 1:1 weighting or any other weighting between 10,000:1 and 1:10,000.

The sequence data used in the k-mer analysis may comprise sequences of TRB and TRG, combined with a 1:1 weighting or any other weighting between 10,000:1 and 1:10,000.

The sequence data used in the k-mer analysis may comprise sequences of TRB and TRD, combined with a 1:1 weighting or any other weighting between 10,000:1 and 1:10,000.

The sequence data used in the k-mer analysis may comprise sequences of any combination of two, three or four of TRA, TRB, TRG and/or TRD, combined using a 1:1 weighting or any other relative weightings.

The sequence data used in the k-mer analysis may comprise sequences of any two of IGH, IGK and/or IGL, combined with a 1:1 weighting or any other weighting between 10,000:1 and 1:10,000.

The sequence data used in the k-mer analysis may comprise of all three of IGH, IGK and/or IGL, combined with a 1:1 weighting or any other weighting between 10,000:1 and 1:10,000.

The sequence data used in the k-mer analysis may comprise sequences of any combination of two or more of TRA, TRB, TRG, TRD, IGH, IGK and/or IGL, combined with a 1:1 weighting or any other relative weightings.

The method of the invention may be carried out in vitro.

The subject may be a mammal and is preferably a human, but may alternatively be a monkey, ape, cat, dog, cow, horse, rabbit or rodent.

According to a further aspect of the present invention there is provided a system for classifying a sample, said system comprising a microprocessor and memory, wherein the sample comprises a T-cell and/or B-cell receptor repertoire, wherein the processor is configured to undertake the method as defined in the preceding aspect.

According to another aspect of the present invention there is provided a computer readable medium storing instructions executable by one or more processors to perform operations according to the method as defined in the preceding aspect.

According to a yet further aspect the invention provides a k-mer dataset produced using the method of the invention, derived from k-mer analysis of sequence data of TRA, TRB, TRG, TRD, IGH, IGK and/or IGL from samples of known disease status, known physiological status or known pathophysiological status, for use as a training or reference data set which can be used to classify new or test samples.

In some example embodiments the set of instructions/method steps described above are implemented as functional and software instructions embodied as a set of executable instructions which are effected on a computer or machine which is programmed with and controlled by said executable instructions. Such instructions are loaded for execution on a processor (such as one or more CPUs). The term processor includes microprocessors, microcontrollers, processor modules or subsystems (including one or more microprocessors or microcontrollers), or other control or computing devices. A processor can refer to a single component or to plural components.

In other examples, the set of instructions/methods illustrated herein and data and instructions associated therewith are stored in respective storage devices, which are implemented as one or more non-transient machine or computer-readable or computer-usable storage media or mediums. Such computer-readable or computer-usable storage medium or media is (are) considered to be part of an article (or article of manufacture). An article or article of manufacture can refer to any manufactured single component or multiple components. The non-transient machine or computer-usable media or mediums as defined herein excludes signals, but such media or mediums may be capable of receiving and processing information from signals and/or other transient mediums.

Example embodiments of the material discussed in this specification can be implemented in whole or in part through network, computer, or data based devices and/or services. These may include cloud, internet, intranet, mobile, desktop, processor, look-up table, microcontroller, consumer equipment, infrastructure, or other enabling devices and services.

The skilled man will appreciate that preferred features of any one embodiment, claim and/or aspect of the invention may be applied to all other embodiments, claims and/or aspects of the invention.

The present invention will be further described in more detail, by way of example only, with reference to the following figures in which:

FIG. 1 illustrates the current method of screening for coeliac disease in an adult.

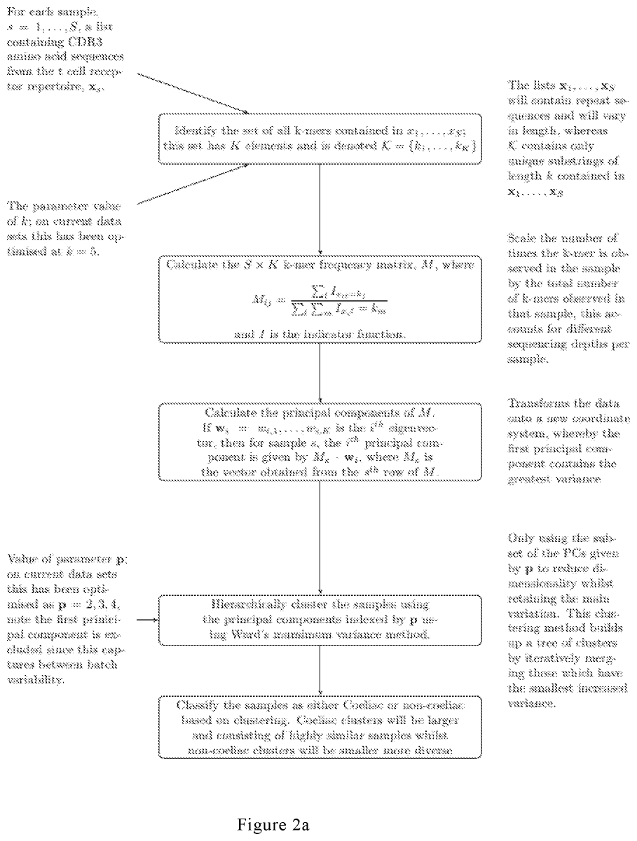

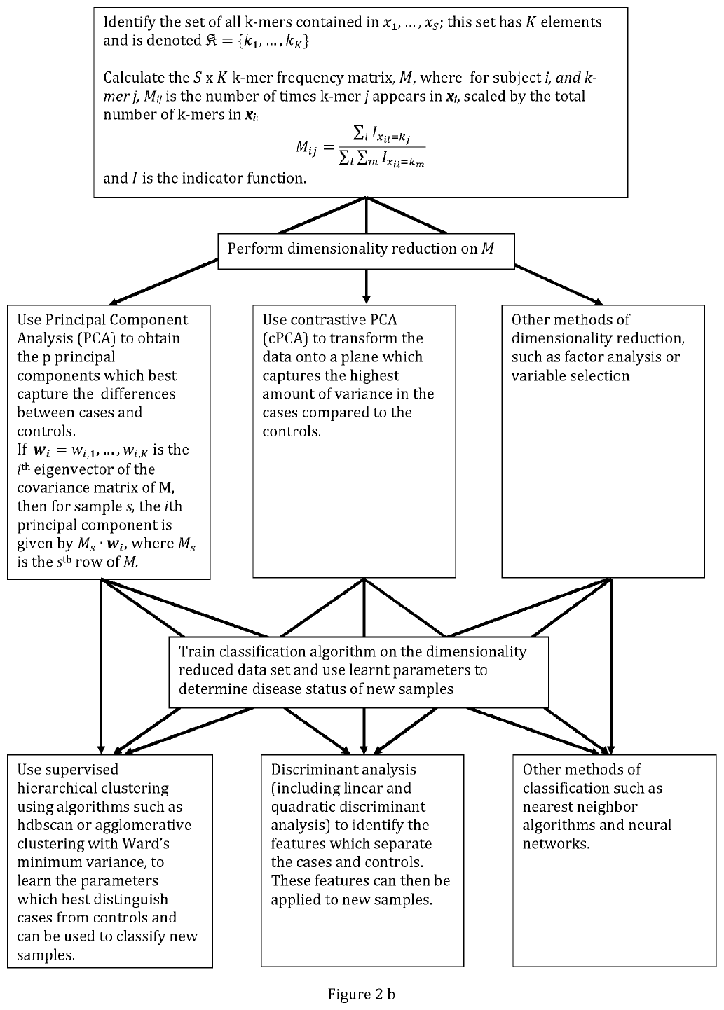

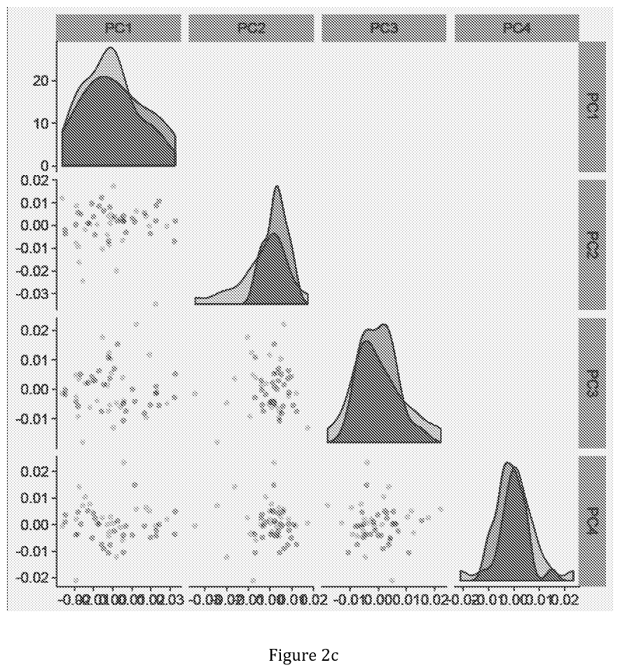

FIG. 2a illustrates an embodiment of the present invention, in particular with reference to the mathematical analysis of the data.

FIG. 2b illustrates a range of embodiments of the present invention, in particular with reference to the mathematical analysis of the data, showing that k-mer analysis is performed followed by a methodology that decreases the dimensions of the data, including but not limited to principal component analysis, and that a classification methodology is used, including but not limited to clustering methods. Dimensionality reduction methods include principal component analysis and contrastive principal component analysis. Methods that can provide both dimensionality reduction and subsequent classification include linear discriminant analysis, generalised discriminant analysis, quadratic discriminant analysis and canonical correlation analysis. Methods that can provide classification, usually following a dimensionality reduction method, include clustering approaches (which may or may not be hierarchical and which can be supervised or unsupervised), support vector machines, logistic regression, nearest neighbour analysis, decision trees and neural network-based approaches. Details of such methodologies may be found in standard mathematical textbooks (Hastie, T; Tibshirani, R; Friedman, J. The elements of statistical learning: Data Mining, Inference, & Prediction. 2nd Edition. Springer, N Y 2009).

FIG. 2c shows an embodiment plotting the values of the first four principal components against each other. These are generated when principal component analysis is carried out on the matrix M. This transforms the data onto a new coordinate system, whereby the first principal component contains the greatest variance. This plot provides an indication of the relative expression of the principal components in relation to each other. From the plot, it can be seen that the greatest separation between batches (light points vs. dark points) is seen in the first principal component labelled PC1 and shown in the scatterplot of PC1 vs. PC2 (the first principal component versus the second principal component. Typically, the first principal component is discarded, as it tends to describe batch variability between samples. Accordingly, the principal components 2 to 10 are generally used for indexing, either individually or as combinations of two or more principal components.

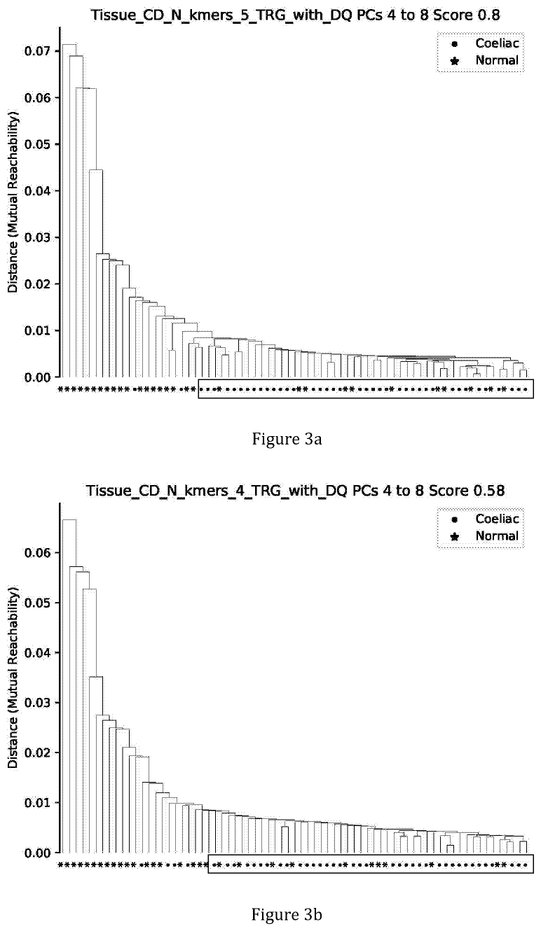

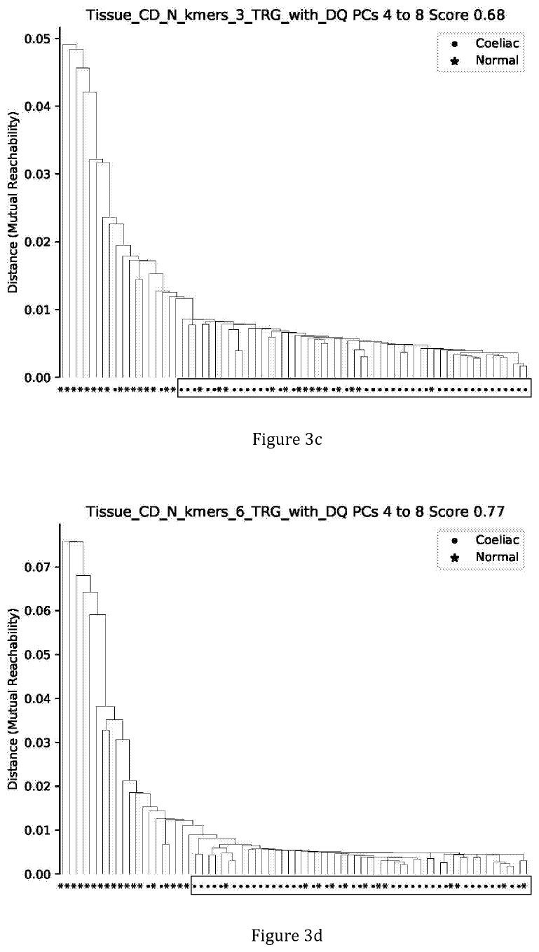

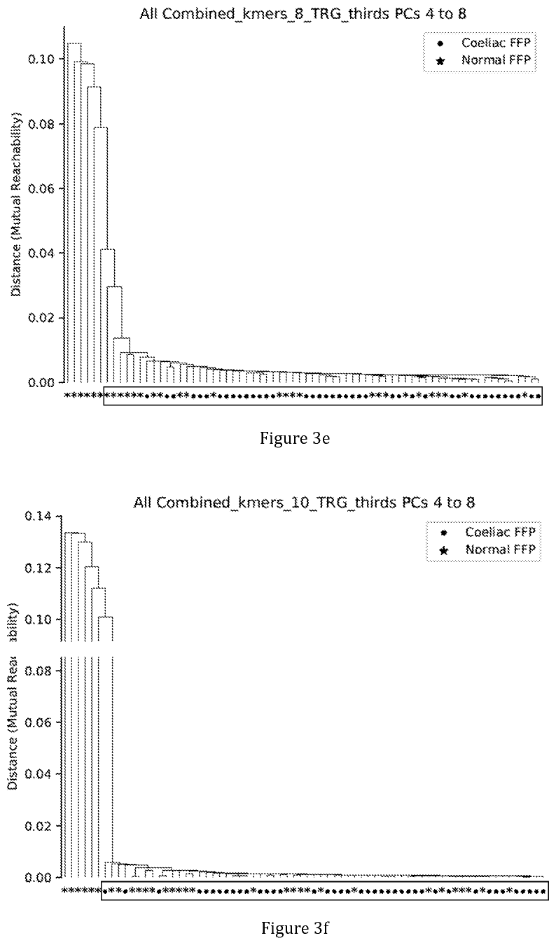

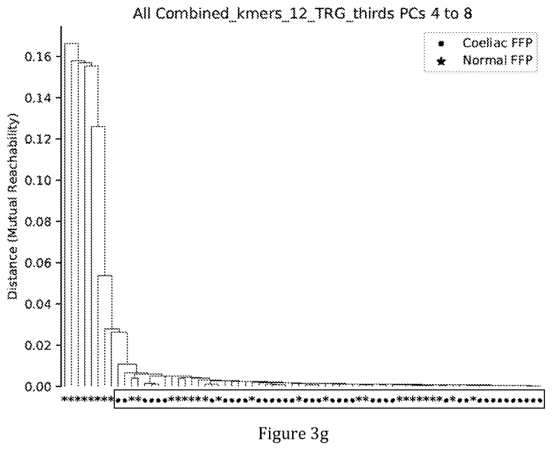

FIG. 3a shows hierarchical clustering of samples (as shown schematically in FIG. 2a) based on principal components 4-8 (those with the highest association with coeliac versus normal samples) of TRG 5-mer frequencies. For all samples analysed by principal component analysis followed by clustering, in FIGS. 3 to 14, the Python programme HDBSCAN (see data analysis section) was used to perform hierarchical clustering. Normal samples are stars; coeliac samples are dots. The samples on the right contain the majority of the coeliac samples whereas the normal samples are on the left. The box shows the area within the cluster plot into which the majority of coeliac samples fall. With a sufficiently sized training set, all new samples falling into such an area might be classified as coeliac samples. Additionally, a score known as “purity” is provided. To generate this purity score, each cluster is given a label (either coeliac or normal) based on which type of sample is more frequent within the cluster. The score is then calculated as the proportion of samples whose label matches its corresponding cluster's label. A purity score of <0.5 would indicate worse than random clustering, a purity score of 0.5 would indicate random clustering, while a purity score between 0.5 and 1.0 would indicate better clustering than expected by random clustering. A purity score in this dendrogram of 0.8 indicates reasonably accurate classification. All sequence data was obtained from formalin fixed paraffin embedded samples. The subjects were classified prior to the k-mer analysis using the “gold standard” of histology and the high purity score of 0.8 indicates a high level of correct diagnosis by the algorithm, using histology as the “gold standard” test for coeliac disease. As this “gold standard” test is far from perfect it is likely that some of the subjects were misdiagnosed and wrongly classified by histology by histology.

FIG. 3b shows clustering of samples (as shown schematically in FIG. 2a), as in FIG. 3a, based on principal components 4-8 of TRG 4-mer rather than 5-mer frequencies, indicating that changing k-mer length affects accuracy of clustering. For an explanation of the box see FIG. 3a. A purity score of 0.58 is observed.

FIG. 3c shows clustering of samples (as shown schematically in FIG. 2a), as in FIG. 3a, based on principal components 4-8 of TRG 3-mer rather than 5-mer frequencies, indicating that changing k-mer length affects accuracy of clustering. This has a purity score of 0.68 indicating that the shorter k-mer length has led to less accurate classification.

FIG. 3d shows clustering of samples (as shown schematically in FIG. 2a), as in FIG. 3a, based on principal components 4-8 of TRG 6-mer rather than 5-mer frequencies, indicating that changing k-mer length affects accuracy of clustering. For an explanation of the box see FIG. 3a. A purity score of 0.77 is observed.

FIG. 3e shows clustering of samples (as shown schematically in FIG. 2a), as in FIG. 3a, based on principal components 4-8 of TRG 8-mer rather than 5-mer frequencies, indicating that changing k-mer length affects accuracy of clustering. For an explanation of the box see FIG. 3a. A purity score of 0.65 is observed.

FIG. 3f shows clustering of samples (as shown schematically in FIG. 2a), as in FIG. 3a, based on principal components 4-8 of TRG 10-mer rather than 5-mer frequencies, indicating that changing k-mer length affects accuracy of clustering. For an explanation of the box see FIG. 3a. A purity score of 0.58 is observed.

FIG. 3g shows clustering of samples (as shown schematically in FIG. 2a), as in FIG. 3a, based on principal components 4-8 of TRG 12-mer rather than 5-mer frequencies, indicating that changing k-mer length affects accuracy of clustering. For an explanation of the box see FIG. 3a. A purity score of 0.65 is observed.

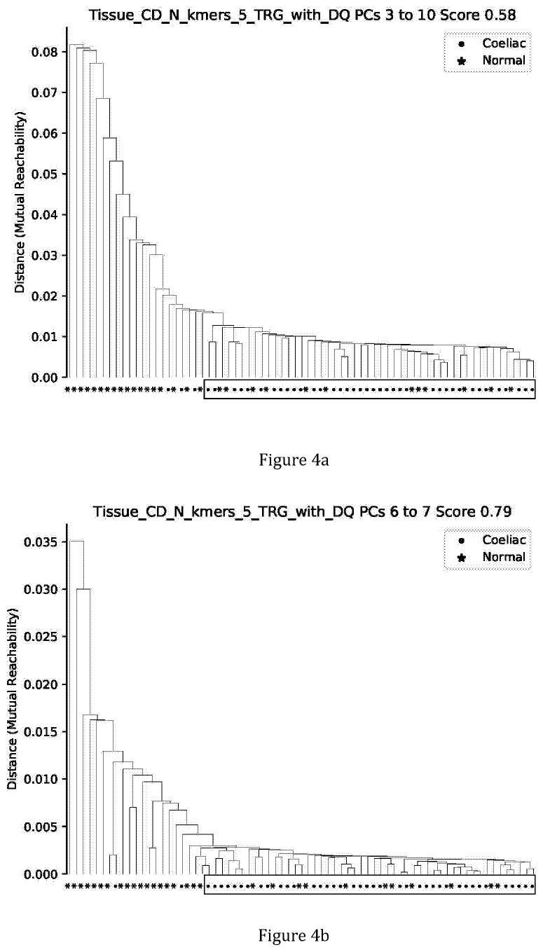

FIG. 4a shows clustering of samples (as shown schematically in FIG. 2a), as in FIG. 3a, based on principal components 3-10 of TRG 5-mer frequencies, indicating that using different principal components affects accuracy of clustering. The purity score of 0.58 indicates suboptimal classification by the algorithm. For an explanation of the box see FIG. 3a.

FIG. 4b shows clustering of samples (as shown schematically in FIG. 2a), as in FIG. 3a, based on principal components 6-7 of TRG 5-mer frequencies, indicating that using different principal components affects accuracy of clustering. For an explanation of the box see FIG. 3a. A purity score of 0.79 indicates more accurate classification using these parameters than those used in FIG. 4a.

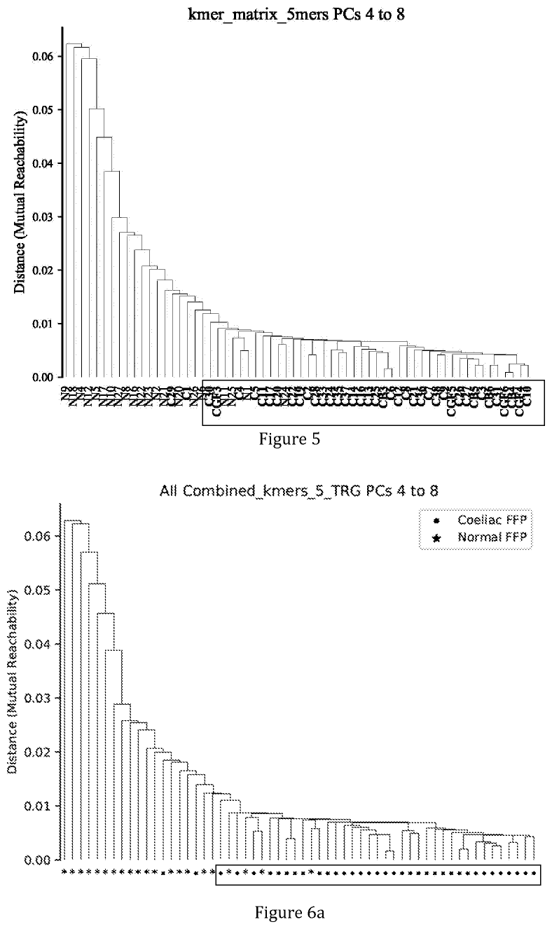

FIG. 5 shows clustering of samples (using principal component analysis, as shown schematically in FIG. 2a) based on principal components 4-8 (those with the highest association with coeliac/normal samples) of TRG 5-mer frequencies. Normal samples are prefixed with “N”; coeliac samples are prefixed with “C”; samples from patients with known coeliac disease on long term (>6 months) gluten-free diet, which showed normal histology, are prefixed with “CGF”. The samples on the right contain the majority of the coeliac samples whereas the normal samples are on the left. For explanation of box, please see FIG. 3a. All sequence data was obtained from formalin fixed paraffin embedded samples. Unlike any other test for coeliac disease (Coeliac disease: recognition, assessment and management; National Institute for Health and Care Excellence (NICE); https://www.nice.org.uk/guidance/ng20/chapter/Recommendations), this algorithm is able to correctly classify (diagnose) samples from patients who were not exposed to gluten prior to biopsy.

FIG. 6a shows clustering of the same samples as shown in FIG. 5 (using principal component analysis, as shown schematically in FIG. 2a) based on principal components 4-8 (those with the highest association with coeliac/normal samples) of TRG 5-mer frequencies, without the 5-mers being annotated as to their position at the beginning, in the middle and at the end of the CDR3 region. For an explanation of the box see FIG. 3a.

FIG. 6b shows clustering of the same samples as shown in FIG. 6a (using principal component analysis, as shown schematically in FIG. 2a) based on principal components 4-8 (those with the highest association with coeliac/normal samples) of TRG 5-mer frequencies, with 5-mers being annotated as to their position at the beginning, in the middle and at the end of the CDR3 region. Annotation of k-mers with their position within CDR3 further improves clustering and such k-mer annotation is amenable to any of the analytical methodologies shown in FIG. 2b. For an explanation of the box see FIG. 3a.

FIG. 7a shows clustering of samples (as shown schematically in FIG. 2a) based on principal components 6-7 (those with the highest association with coeliac/normal samples) of TRD 4-mer frequencies. Normal samples are prefixed with “N”; coeliac samples are prefixed with “C”. The samples on the right contain the majority of the coeliac samples whereas the normal samples are on the left. All sequence data was obtained from formalin fixed paraffin embedded samples. The subjects were classified prior to the k-mer analysis using histology as the “gold standard” test for coeliac disease. As this “gold standard” test is far from perfect it is possible that one or more of the subjects were misdiagnosed and wrongly classified by histology.

FIG. 7b shows clustering of samples (as shown schematically in FIG. 2a) based on principal components 1-10 (those with the highest association with coeliac/normal samples) of TRB 4-mer frequencies. Normal samples are shown as +; coeliac samples are shown as dots. The samples on the right contain the majority of the coeliac samples whereas the normal samples are on the left. For an explanation of the box see FIG. 3a. All sequence data was obtained from formalin fixed paraffin embedded samples. The subjects were classified prior to the k-mer analysis using histology as the “gold standard” test for coeliac disease. As this “gold standard” test is far from perfect it is possible that one or more of the subjects were misdiagnosed and wrongly classified by histology. The purity score of 0.62 essentially indicates that classification by the algorithm could be improved. Analysis of data from multiple different TCR loci as shown in FIGS. 3a, 7a and 7b indicates that data from multiple TCR and/or immunoglobulin loci could be combined (FIG. 7d) to further refine sample classification.

FIG. 7c shows clustering of samples (as shown schematically in FIG. 2a) based on principal components 1-10 (those with the highest association with coeliac/normal samples) of TRB 4-mer frequencies. Normal samples are shown as +; coeliac samples are shown as dots. Only HLA-DQ2+DQ8− coeliac samples and control samples have been used in this analysis. The samples on the right contain the majority of the coeliac samples whereas the normal samples are on the left. For explanation of box, please see FIG. 3a. All sequence data was obtained from formalin fixed paraffin embedded samples. The subjects were classified prior to the k-mer analysis using histology as the “gold standard” test for coeliac disease. As this “gold standard” test is far from perfect it is possible that one or more of the subjects were misdiagnosed and wrongly classified by histology. The purity score of 0.83 for HLA-DQ2+DQ8-matched coeliac and normal samples, compares favourably with a purity score of 0.62 when samples are clustered without taking HLA type into consideration, indicating that prior stratification of samples by HLA type may be useful in coeliac disease and possibly in other conditions.

FIG. 7d shows clustering of the samples used in FIG. 7a based on principal components 1 and 2 (those with the highest association with coeliac/normal samples) of TRD and TRG 3-mer frequencies, giving a 4:1 weighting (or using a 4:1 ratio) of TRD to TRG sequence data. Normal samples are prefixed with “N”; coeliac samples are prefixed with “C”. The samples on the left contain the majority of the coeliac samples whereas the normal samples are on the right. All sequence data was obtained from formalin fixed paraffin embedded samples. The subjects were classified prior to the k-mer analysis using histology as the “gold standard” test for coeliac disease. As this “gold standard” test is far from perfect it is possible that one or more of the subjects were misdiagnosed and wrongly classified by histology.

FIG. 8 shows clustering of samples (as shown schematically in FIG. 2a) based on principal components 5-7 (those with the highest association with coeliac/normal samples) of TRG 5-mer frequencies, demonstrating the applicability of this approach to fresh tissue, as well as formalin fixed paraffin embedded tissue. Formalin fixed paraffin embedded normal samples are +signs and fresh normal samples are vertical lines; formalin fixed paraffin embedded coeliac samples are dots and fresh coeliac samples are stars. The samples on the right contain the majority of the coeliac samples whereas the normal samples are on the left. While the clustering of the fresh samples with the formalin fixed samples is less strong than for formalin fixed samples alone, this is likely to be due to variation in sequencing depth between the two tissue types.

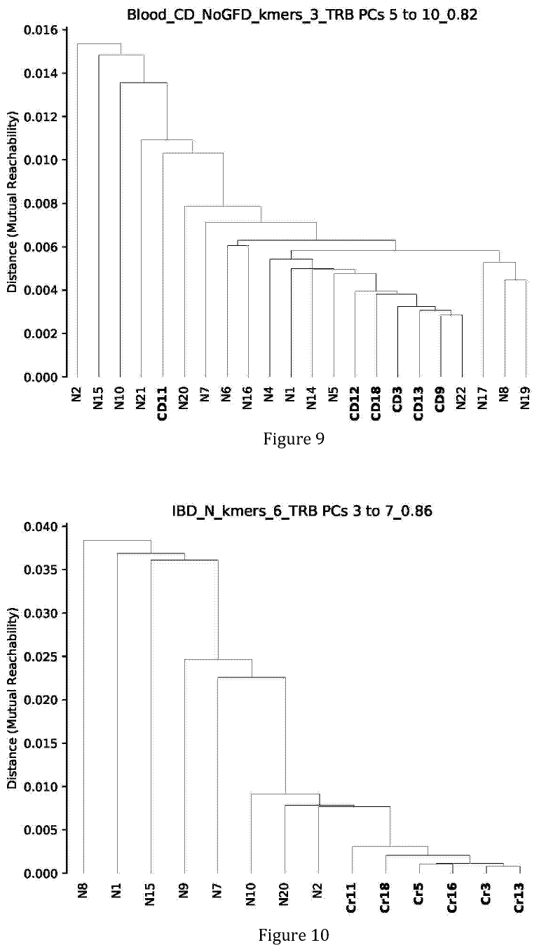

FIG. 9 shows clustering of samples (as shown schematically in FIG. 2a) based on principal components 5-10 (those with the highest association with coeliac/normal samples) of TRB 3-mer frequencies, demonstrating the applicability of this approach to blood samples. The purity score of 0.82 indicates reasonably accurate clustering of samples.

FIG. 10 shows clustering of samples (as shown schematically in FIG. 2a) based on principal components 3-7 (those with the highest association with Crohn's disease/normal samples) of TRB 6-mer frequencies. Normal samples are prefixed with “N”; Crohn's disease samples are prefixed with “Cr”. All the samples on the right are Crohn's disease samples whereas all the normal samples are on the left. All sequence data was obtained from formalin fixed paraffin embedded samples. The subjects were classified prior to the k-mer analysis using the “gold standard” of histology and the purity score of 0.86 essentially indicates accurate clustering of samples by the algorithm.

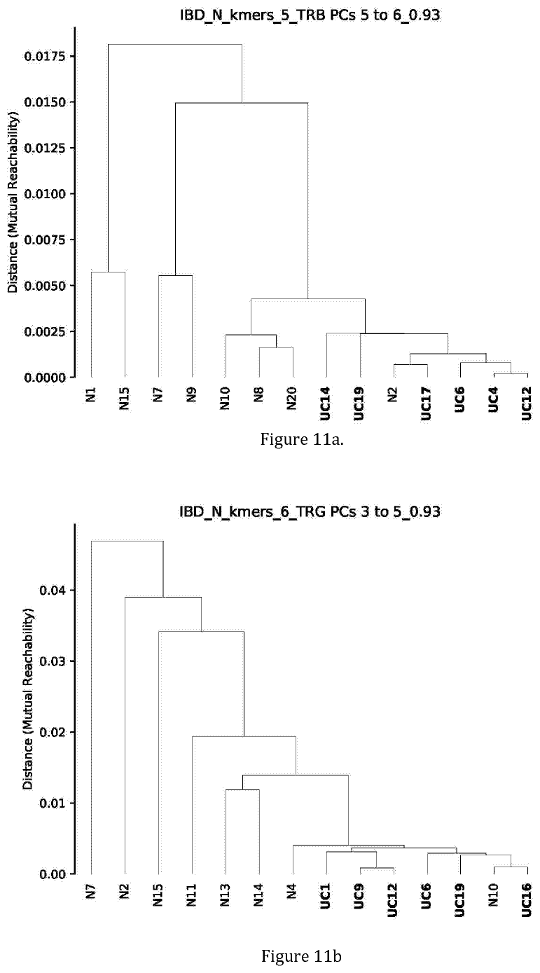

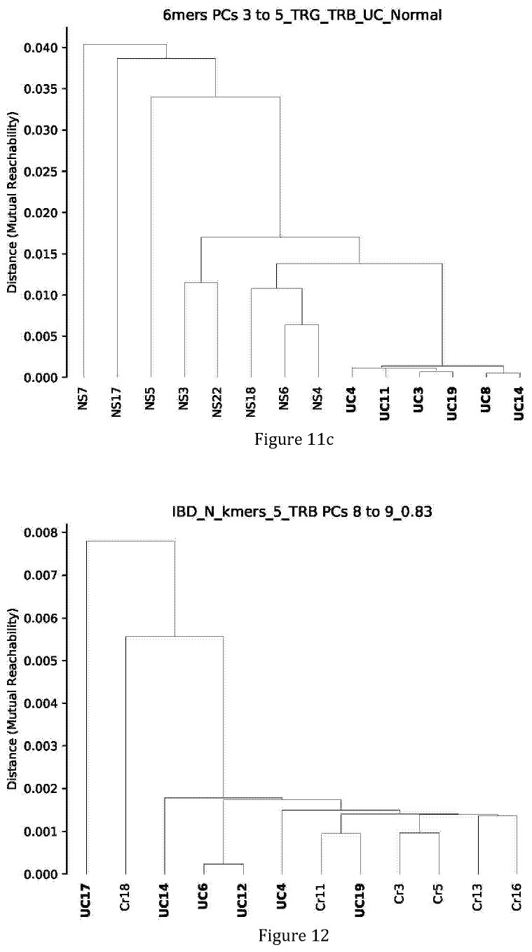

FIG. 11a shows clustering of samples (as shown schematically in FIG. 2a) based on principal components 5-6 (those with the highest association with ulcerative colitis/normal samples) of TRB 5-mer frequencies. Normal samples are prefixed with “N”; Ulcerative colitis samples are prefixed with “U”. Almost all the samples on the right are ulcerative colitis samples, whereas the normal samples are on the left. All sequence data was obtained from formalin fixed paraffin embedded samples. The subjects were classified prior to the k-mer analysis using the “gold standard” of histology and the purity score of 0.93 essentially indicates accurate clustering of samples by the algorithm.

FIG. 11b shows clustering of samples (as shown schematically in FIG. 2a) based on principal components 3-5 (those with the highest association with ulcerative colitis/normal samples) of TRG 6-mer frequencies. Normal samples are prefixed with “N”; Ulcerative colitis samples are prefixed with “U”. Almost all the samples on the right are ulcerative colitis samples, whereas the normal samples are on the left. All sequence data was obtained from formalin fixed paraffin embedded samples. The subjects were classified prior to the k-mer analysis using the “gold standard” of histology and the purity score of 0.93 indicates accurate clustering of samples by the algorithm. It can be appreciated by the skilled person that amalgamating the datasets in FIGS. 11a and 11b could further improve classification of the samples.

FIG. 11c shows clustering of samples (as shown schematically in FIG. 2a) based on principal components 3-5 (those with the highest association with ulcerative colitis/normal samples) of a combined analysis of TRB and TRG 6-mer frequencies, with a 1:1 weighting of TRB:TRG k-mers (that is, using a 1:1 ratio of TRB:TRG k-mers in the analysis). Normal samples are prefixed with “NS”; Ulcerative colitis samples are prefixed with “U”. All the samples on the right are ulcerative colitis samples, whereas the normal samples are on the left. All sequence data was obtained from formalin fixed paraffin embedded samples. The subjects were classified prior to the k-mer analysis using the “gold standard” of histology. This demonstrates that amalgamating the datasets in FIGS. 11a and 11b (i.e., using k-mer data from 2 loci) can further improve classification of the samples and from this it can be appreciated that amalgamation of data from 3 or more loci might also improve sample classification further in certain situations.

FIG. 12 shows clustering of samples (as shown schematically in FIG. 2a) based on principal components 8-9 (those with the highest association with ulcerative colitis/Crohn's disease samples) of TRB 5-mer frequencies. Ulcerative colitis samples are prefixed with “U”; Crohn's disease samples are prefixed with “Cr”. Almost all the samples on the right are Crohn's disease samples whereas the ulcerative colitis samples are on the left. All sequence data was obtained from formalin fixed paraffin embedded samples. The subjects were classified prior to the k-mer analysis using the “gold standard” of histology and the purity score of 0.83 essentially indicates reasonably accurate clustering of samples by the algorithm.

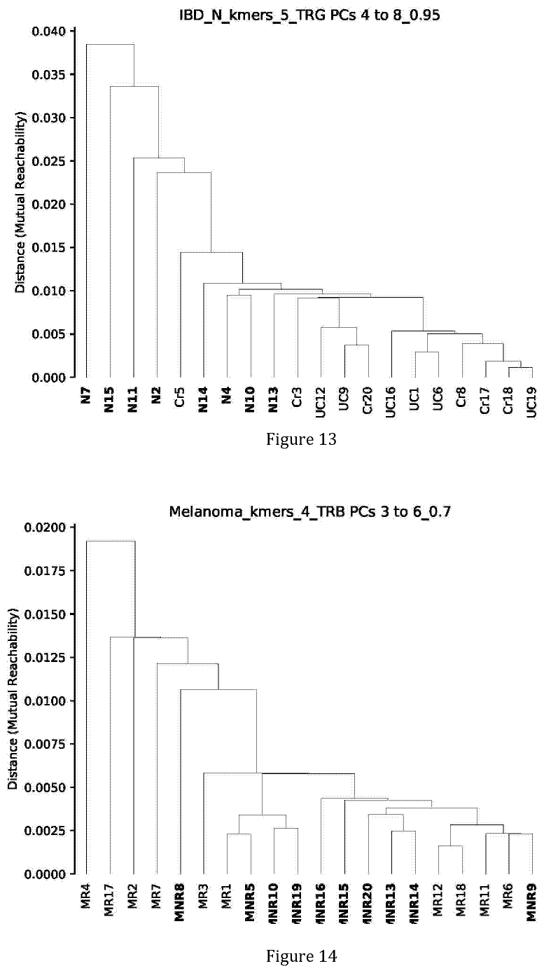

FIG. 13 shows clustering of samples (as shown schematically in FIG. 2a) into three separate groups based on principal components 4-8 (those with the highest association with normal/ulcerative colitis/Crohn's disease samples) of TRG 5-mer frequencies. Normal samples are prefixed with “N”; ulcerative colitis samples are prefixed with “U”; Crohn's disease samples are prefixed with “Cr”. Almost all the samples on the right are Crohn's disease and ulcerative colitis samples, whereas normal samples are on the left. All sequence data was obtained from formalin fixed paraffin embedded samples. The subjects were classified prior to the k-mer analysis using the “gold standard” of histology and the purity score of 0.95 indicates highly accurate clustering of abnormal (ulcerative colitis and Crohn's disease) and normal samples into separate groups. This plot indicates the potential for separation of samples into more than two groups.

FIG. 14 shows clustering of samples (as shown schematically in FIG. 2a) based on principal components 3-6 (those with the highest association with melanoma patient samples in whom recurrence and metastasis occurred (prefixed MR) ulcerative colitis/with melanoma patient samples in whom no recurrence or metastasis occurred (prefixed MR) within a two year follow-up period) of TRB 4-mer frequencies. The purity score of 0.7 essentially indicates moderately good separation of samples. All sequence data was obtained from formalin fixed paraffin embedded samples of sentinel lymph nodes, i.e., the nearest draining lymph node to the site of the melanoma.

FIG. 15 shows the utility of contrastive principal component analysis followed by clustering of samples (as shown schematically in FIG. 2b) based on contrastive principal components 2-7 (those with the highest association with coeliac/normal samples) of TRG 5-mer frequencies on a subset of samples shown in FIG. 3a. For the cPCA, the parameters were: number_of_alphas=50; maximum_log_alpha=50. The background data set consisted of the samples: N2, N20, N11, N12, N16, N17, N18, N2, N20, N21, while the foreground data set consisted of the samples: C, C10, C11, C12, C13, C14, C16, C17, C19, C2, C20, N1, N10, N22, N23, N24, N25, N26, N27, N4, N5, N6, N8, N9. As for samples in which principal component analysis and clustering are shown (FIGS. 3 to 14), the Python programme HDBSCAN (see data analysis section) was used to perform hierarchical clustering. Normal samples are prefixed “N”; coeliac samples are prefixed “C”. The samples on the right contain the majority of the coeliac samples whereas the normal samples are on the left. All sequence data were obtained from formalin fixed paraffin embedded samples. The subjects were classified prior to the k-mer analysis using the “gold standard” of histology. As this “gold standard” test is far from perfect it is likely that some of the subjects were misdiagnosed and wrongly classified by histology.

FIG. 16 shows the accuracy score output for the classification of samples (as shown schematically in FIG. 2b) based on the principal components stated for TRG5-mer frequencies following analysis by quadratic discrimination analysis (QDA), using a subset of the data presented in FIG. 3a. Following principal component analysis, we fitted a QDA model with the coeliac disease and normal training samples (see data analysis section for further details) and used it to predict the classes of both the training samples and the new (test) samples being added (in this case new coeliac disease samples), as shown in FIGS. 17a-17d. As can be seen in the columns headed, “new sample prediction accuracy”, all new (test) samples added were classified correctly. The “accuracy score” refers to the accuracy of the prediction for the training sample set and is the total number of correct predictions divided by the total number of predictions. The “overall accuracy” refers to the accuracy of the prediction for the training and test sample sets and is the total number of correct predictions divided by the total number of predictions. “Positional annotation” of k-mers refers to annotating them with information about whether they were derived from the beginning, middle or end of the CDR3 region.

FIG. 17a shows the predicted diagnoses of training set patient samples on the basis of TRG 5-mer frequencies without positional annotation, based on principal components 4 and 5 (x=predicted coeliac samples; dot=predicted normal samples). See also FIG. 16. Only 2 dimensional plots are possible in this document, but FIG. 16 indicates the accuracy of sample classification based on a multidimensional space analysis, involving more than two principal components. An approach in multidimensional space is able to classify samples based on more than two principal components and thus gives a more accurate classification than plots, such as this and FIGS. 17b-17d, which are based on the consideration of only two principal components.

FIG. 17b shows the actual diagnoses of training set patient samples on the basis of TRG 5-mer frequencies without positional annotation, based on principal components 4 and 5 (x=predicted coeliac samples; dot=predicted normal samples; *—new sample (actually known coeliac disease) being added). See also FIGS. 16 and 17a.

FIG. 17c shows the predicted diagnoses of training set patient samples on the basis of TRG 5-mer frequencies, with k-mers annotated by position (beginning, middle or end) within CDR3, based on principal components 4 and 5 (x=predicted coeliac samples; dot=predicted normal samples). See also FIGS. 16 and 17a.

FIG. 17d shows the actual diagnoses of training set patient samples on the basis of TRG 5-mer frequencies, with k-mers annotated by position (beginning, middle or end) within CDR3, based on principal components 4 and 5 (x=predicted coeliac samples; dot=predicted normal samples; *—new sample (actually known coeliac disease) being added). See also FIGS. 16 and 17a.

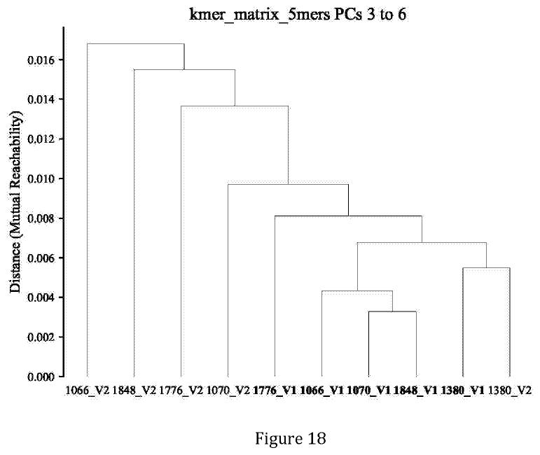

FIG. 18 shows clustering of samples (as shown schematically in FIG. 2a) based on principal components 3-6 (those with the highest association with pre- and post-hepatitis vaccination samples of B-cells from human peripheral blood) of immunoglobulin heavy chain 5-mer frequencies. Samples with a V1 suffix are pre-vaccination and V2 suffix post-vaccination.

Materials and Methods for Disease Status Classification Test

Sample Selection

Up to 60 formalin fixed paraffin embedded (FFPE) biopsy-proven cases of coeliac disease (with histological features categorised as Marsh classification 3B or 3C) and 45 control cases (no histological features of coeliac disease, no history of anaemia or abdominal bloating, biopsy taken for suspected gastro-oesophageal reflux disease) were identified in the diagnostic archive of the Cellular Pathology Department, Oxford University Hospitals NHS Foundation Trust and the Cambridge Human Research Tissue Bank. The wax block containing the formalin fixed paraffin embedded (FFPE) tissue duodenal (biopsy sample) for each case was obtained with full ethical approval from the National Research and Ethics Service (NRES committee South Central, Oxford, reference 04/Q1604/21, P.I. Dr E. Soilleux) and governance approval from the Oxford Radcliffe Hospitals NHS Trust. 5-10 sections of 5 micron thickness were cut from the blocks. Similarly, 10 FFPE sections of sentinel lymph node were cut from 10 melanoma cases without metastasis or recurrence (after 2 years' follow-up) and 10 melanoma cases with both metastasis and recurrence (after 2 years' follow-up). 12 FFPE cases of colonic Crohn's disease, 12 FFPE cases of colonic ulcerative colitis and 12 cases of FFPE normal colon were also obtained.

Fresh (rather than FFPE) duodenal biopsy samples (9 coeliac disease on a gluten-containing diet, 3 coeliac disease on a gluten-free diet and 2 normal) were obtained from Cambridge University Hospitals NHS Foundation Trust and University Hospitals NHS Foundation Trust. Whole blood samples (7 coeliac disease on a gluten-containing diet, 1 coeliac disease on a gluten-free diet and 16 normal) were obtained from Cambridge University Hospitals NHS Foundation Trust or Oxford University Hospitals NHS Foundation Trust. Peripheral blood mononuclear cells were either extracted by density gradient centrifugation over Ficoll or the entire blood sample was lysed for DNA extraction.

Nucleic Acid Extraction

DNA was extracted from ten 5 micron sections of each FFPE biopsy sample, sections having been cut with completely cleaned apparatus between cases to avoid cross-contamination. DNA was extracted using the Generead DNA FFPE kit (Qiagen) as per the manufacturer's instructions. For blood and fresh duodenal samples, DNA was extracted using appropriate Qiagen kits. In the method of the invention either genomic DNA or RNA can be used, and can be extracted using standard techniques.

T-Cell Receptor Repertoire Sequencing

Amplification and Sequencing of the TCR Gamma and Beta Locus

The CE marked kits “LymphotrackR Dx TRG assay panel—PGM”, “LymphotrackR Dx TRG assay panel—MiSeq” (Invivoscribe) or “LymphotrackR Dx TRB assay panel—MiSeq” (Invivoscribe) were used to amplify 210 ng of DNA from each sample, as per the manufacturer's instructions, thus generating a next generation sequencing library ready to run on a an ion PGM™ system for next generation sequencing (Thermo Fisher) or MiSeq 500v2 (Illumina). Briefly, the TRB and TRG kits amplify at least 95% of all the possible V-D-J or V-J rearrangements across the TCR beta or gamma locus in a single round of PCR amplification, simultaneously adding adaptors and indices.

PCR Amplification of the TCR Beta and Delta Loci

Amplification of 200 ng of DNA each for TRB and TRD (or as much remaining DNA as possible, where DNA quantity was limited) was undertaken using the following CE-marked Invivoscribe kits: “TRB gene clonality assay—ABI fluorescence” and “TRD gene clonality assay—ABI fluorescence”. These kits amplify at least 95% of all the possible V-D-J rearrangements across the TCR beta and delta loci, respectively, in a single round of PCR amplification. Three multiplexed PCR tubes are used for the TCR beta amplification and one for the TCR delta amplification. The PCR product in the three TCR beta tubes was pooled into a single tube for next generation sequencing library preparation.

Next Generation Sequencing of the TCR Beta and Delta Loci PCR Products

PCR products were cleaned with Ampure^{XP} beads (Beckman Coulter) and the Kapa Hyper prep kit (KapaBiosystems) used for next library preparation. Sequencing was undertaken on a MiSeq 500v2 (Illumina).

Obtaining RNA Data for Individuals Undergoing Vaccination Against Hepatitis B

RNA data for blood from individuals undergoing vaccination against hepatitis B was obtained from a public, online database (Galson J D, Truck J, Fowler A, Clutterbuck E A, Münz M, Cerundolo V, Reinhard C, van der Most R, Pollard A J, Lunter G, Kelly D F. Analysis of B Cell receptor repertoire Dynamics Following Hepatitis B Vaccination in Humans, and Enrichment of Vaccine-specific Antibody Sequences. EBioMedicine.2015 Nov. 24; 2(12):2070-9). These data had been generated by others from RNA extracted from peripheral blood mononuclear cells (PBMCs), isolated from blood by density-gradient centrifugation over lymphoprep (Asis-Shield Diagnostics). B cells were enriched from PBMCs using CD19 microbeads (Miltenyi Biotec), and the AutoMACS Pro cell separator, and counted using a hemocytometer. 500,000 B cells were isolated for sequencing the total repertoire. In the vaccine group, remaining B cells were labelled with Live/dead-Aqua, CD19-FiTC, CD20-APCH7, CD27-PECy7, CD38-PE, HLA-DR-PerCpCy5 and HBsAg-APC. Viable, CD19+, CD20+, HBsAg+ B cells and viable CD19+, CD20−, CD27+, CD38+, HLA-DR+PCs were then isolated using a MoFlo cell sorter (Beckman Coulter). RNA was extracted from sorted cells using the RNeasy Mini Kit (Qiagen), and reverse transcription performed using SuperScript III (Invitrogen), and random hexamer primers (42° C. for 60 min, 95° C. for 10 min). PCR was conducted using the Multiplex PCR kit (Qiagen), and 200 nM VH-family specific forward primers, with IgM and IgG-specific reverse primers in separate reactions (Wu Y.-C., Kipling D., Leong H. S. High-throughput immunoglobulin repertoire analysis distinguishes between human IgM memory and switched memory B-cell populations. Blood. 2010; 116(7):1070-1078) (94° C. for 15 min, 30 cycles of 94° C. for 30 s, 58° C. for 90 s and 72° C. for 30 s, and 72° C. for 10 min). Amplicons were gel-extracted and purified prior to MiSeq library preparation. Samples were multiplexed, and sequenced across four 2×300 bp MiSeq runs. Sequences from each input sample were de-multiplexed, and paired ends joined using fastq-join (ea-utils).

HLA Typing of Coeliac Disease Samples

30 ng of DNA per sample was submitted for HLA-DQ typing by Q-PCR on a fee-for-service basis to the Molecular Diagnostics Department, Royal Surrey County Hospital, Guildford, U.K., by means of the HLA-DQ (coeliac disease) real-time PCR kit (AnDiaTec, Germany). This specifically determines whether or not the patient possesses the HLA-DQ2 and/or HLA-DQ8 alleles.

Data Analysis

Sequence data was translated into the amino acid sequence in the appropriate reading frame, using information taken from the IMGT database (www.imgt.org) and all sequences containing stop codons were discarded. For the analysis of the T-cell and/or B-cell receptor (TCR and/or BCR) repertoire data, a holistic approach was taken, considering the entire repertoire rather than individual sequences or motifs. For identification of CDR3 regions, in-house methodologies or one of the following approaches were employed: IMGT/V-QUEST (http://www.imgtorg/IMGTindex/V-QUESTphp), http://www.imgt.org/IMGTindex/IM GTHiighV-QUEST.php) IMGT/highV-QUEST or MIXCR (Bolotin, D. A. et al. Nat Methods. 2015 May; 12(5):380-1. MiXCR: software for comprehensive adaptive immunity profiling; https://mixcr.readthedocs.io/en/master/). Using the CDR3 region, all amino acid k-mers (substrings of length K) in the data set were identified, and their frequencies calculated in each sample. These k-mer frequencies are high dimensional and highly correlated; therefore principal component analysis, which is a mathematical transformation that reduces the dimensions of the data whilst capturing the major sources of variation, was performed. Based on the principal components, the data are clustered using the Python package, HDBBSCAN (https://hdbscanreadthedocs.io/en/Iatest/#1), ultimately placing closest together sample that are the most similar. The coeliac samples form a single cluster with high levels of similarity whilst the healthy samples from several smaller clusters with lower levels of similarity. This reflects the belief that the TCR repertoires of coeliac samples are likely to exhibit similar k-mer patterns, whilst healthy samples demonstrate more diverse k-mer patterns. k-mer lengths can be varied, according to the dataset being analysed. In the present analysis, k-mer lengths of 5 were used, because it was determined empirically that this length gave optimum sensitivity and specificity. Different k-mer lengths can be used in the analysis of different autoimmune diseases, following empirical determination of the best k-mer length to separate the biological conditions of interest, and altering k-mer length in the analysis of one specific condition can alter sensitivity and specificity. Other methods of dimensionality reduction can also be used as an alternative to PCA, in particular contrastive PCA which captures the highest variance in a case set relative to controls. Different methods of classification were also used, in particular quadratic discriminant analysis (QDA) using a publically available Python package (http://scikit-learn.org/stable/modules/lda_qda.html; Machine learning in Python, Pedregosa et al., JMLR 12, pp. 2825-2830, 2011), which separates cases and controls using a quadratic surface. These alternative methods of dimensionality reduction and classification, detailed in FIG. 2b, were shown to generate similar results. Various input data can be used with QDA. In generating FIGS. 16 and 17, we chose to use principal components derived from the k-mer analysis in a manner analogous to FIGS. 3 to 14 and the principal components were essentially plotted against each other in multidimensional space for the test set samples and regions in that space defined as containing coeliac and normal samples on the basis of the training set. This then permitted new samples to be classified by being placed into multidimensional space in either a coeliac or a normal region. Obviously, only 2 dimensional plots (shown for QDA in FIG. 17) are possible in this document, but FIG. 16 indicates the accuracy of sample classification based on a multidimensional space analysis, involving more than two principal components

Use of Method of the Invention in Diagnosis

In one embodiment, a database of the TCR repertoires of samples (derived from duodenum or blood or other sites) from patients with coeliac disease and those who do not have the condition is prepared. Test samples are then compared to these databases using the method of the invention. Preferably the comparison is fully automated, comparing each test sample with the samples in the database. The exact point of clustering of the test sample within the reference samples (either with coeliac disease samples or unaffected samples) allows the diagnosis to be made.

FIG. 2a provides an overview of an embodiment of the present invention. In the example shown, each sample (s=1 . . . , S) is analysed and a list containing CDR3 amino acid sequences obtained from the t cell receptor repertoire xₛ. A set K of all k-mers contained within x₁, . . . , xₛ, containing K elements is denoted by K={k₁, . . . , k_{K}} can also be computed.

As noted in FIG. 2a, The lists x₁, . . . , xₛ will contain repeat sequences and will vary in length, whereas K contains only unique substrings of length k contained in xᵢ, . . . , xₛ.

In order to normalize the k-mer frequency data, the number of times that the k-mer is observed in the sample is then scaled by the total number of k-mers observed in that sample. This accounts for different sequencing depths per sample.

Sequencing depths typically are around 100,000 sequences, but a depth of 50,000 is typically sufficient to allow analysis. It can be appreciated that utilising a higher sequencing depth is typically preferable; depths of over 330,000 have been utilised in the present method, however some samples may be of insufficient quality to provide such a high number.

The normalization is the calculation of a frequency matrix describing the relative total number of k-mers observed in the sample. The frequency matrix scales the number of times a k-mer is observed in a sample by the total number of k-mers observed in the sample for each k-mer and for each sample.

The frequency matrix, a S×K k-mer frequency matrix, M, is then calculated:

$${M}_{i , j} = \frac{{Σ}_{l} {I}_{{x}_{ii} = {k}_{j}}}{{Σ}_{l} {Σ}_{m} {I}_{xil} = {k}_{rx}}$$

where Iₓᵢₗ₌ₖⱼ is an indicator function which takes the value 1 when xᵢₗ=kⱼ and 0 otherwise (where xᵢₗ is the l^{th} k-mer in sample i and kⱼ is the j^{th} k-mer in the set K, as defined above).

Principal component analysis is then carried out on the matrix M. This transforms the data onto a new coordinate system, whereby the first principal component contains the greatest variance. FIG. 2c provides an indication of the relative expression of the principal components in relation to each other. From FIG. 2c, it can be seen that the greatest separation between batches (light points vs. dark points) is seen in the first principal component labelled PC1 and shown in the scatterplot of PC1 vs. PC2 (the 1^{st} principal component vs. the 2^{nd} principal component. Typically, the first principal component is discarded, as it tends to describe batch variability between samples. Accordingly, the principal components 2 to 10 are generally used for indexing, either individually or as combinations of two or more principal components.

In the embodiment described herein, for the frequency matrix M, if wᵢ=w_{i,1}, . . . , w_{i, K} is the i^{th} eigenvector, then for samples, the i^{th} principal component is given by Mₛ·wᵢ, where Mₛ is the vector obtained from the s^{th} row of M.

The principal component data is then clustered using hierarchical or other means of clustering, which may be supervised or unsupervised. The result is that coeliac samples form a single cluster with high levels of similarity whilst the healthy samples from several smaller clusters with lower levels of similarity. This reflects the understanding that the TCR repertoires of coeliac samples exhibit similar k-mer patterns, whilst healthy samples are more diverse.

Clustering is undertaken using the principal components indexed according to a parameter p, where p is the principal component, and p=2, 3, 4. Ward's minimum variance method is one option that can be used for the clustering, although the skilled person would appreciate that alternatives exist.

From this analysis, samples are then indexed, meaning that they are grouped or clustered based on their relative variance or principal components. Samples having the smallest increased variance are then merged. New, test samples can then be assigned to clusters which are classed as either coeliac or normal, in order to determine their disease status.

Additionally, as shown in FIG. 2b, alternative methods of dimensionality reduction, including contrastive principal component analysis (cPCA) and feature selection (also known as variable selection), could be used. Further, alternative methods of classification may be used, usually following a dimensionality reduction method. These include clustering approaches (which may or may not be hierarchical and which can be supervised or unsupervised), support vector machines, logistic regression, nearest neighbour analysis, decision trees and neural network-based approaches. Certain methods can provide both dimensionality reduction and subsequent classification, for example linear discriminant analysis, generalised discriminant analysis, quadratic discriminant analysis and canonical correlation analysis. Details of such methodologies may be found in standard mathematical textbooks (Hastie, T; Tibshirani, R; Friedman, J. The elements of statistical learning: Data Mining, Inference, & Prediction. 2nd Edition. Springer, N Y 2009). Test samples can then be assigned a disease status based on the optimal parameters for these methods, the optimal parameters having been determined by application of the method to a training set of data from samples with known disease status.

FIGS. 3 to 15 and 18 show dendrograms that display how samples having ‘known’ disease or outcome status (as determined using the “gold standard” of histology or clinical follow up, respectively) cluster when subject to analysis using the present invention. Samples are clustered according to the methodology described above, particularly with reference to FIGS. 2a and 2b. The y-axis describes mutual reachability, a measure of similarity between clusters by showing a relative distance between k-mer frequency data points. Therefore, a high distance between points is indicative of a low likelihood of data clustering or correlating.

Each cluster is grouped and further similarity analysis undertaken. From the results, it can be seen that each group or cluster provides a cut-off value, which allows a diagnosis to be made. For example, for a k-mer length of 5 (FIG. 5), a mutual reachability value of 0.01 would allow a diagnosis of coeliac to be made.

Each new sample or cohort added to the ensemble or collection of existing results requires a fresh calculation, however the clustering and the cut-off values may be used to provide diagnosis of the new sample.

In practice the method of the invention would be used in a clinical setting. More specifically a patient with abdominal pain, bloating, fatigue who is shown to be anaemic would have a sample taken, either a biopsy or blood, which would then be analysed by the method of the invention and a ratio, percentage or relative likelihood of the subject having coeliac disease determined. A ratio, percentage or relative likelihood, above which a firm diagnosis of coeliac disease should be made, might be defined on an empirical basis for the test under particular conditions. If necessary the patient would be put on a gluten free diet.

In addition to clustering using TRG, clustering using TRD and TRB can be used to separate FFPE duodenal samples from coeliac patients from those obtained from normal patients (FIG. 3a), the TRB data being more discriminating if compared within HLA-DQ class (e.g., all HLA-DQ2; (as shown in FIG. 7c, compared with samples compared across all HLA-DQ classes in FIG. 7b).

To demonstrate the utility of this methodology in coeliac disease diagnosis, regardless of whether or not the patient is on a gluten-containing diet, we further demonstrated that TRG data from duodenal biopsies, which appear histologically normal, from patients with coeliac disease on a gluten-free diet (GFD) clusters with TRG data from patients with coeliac disease rather than from duodenal biopsies from patients without coeliac disease (FIG. 5). To demonstrate the applicability of the method to other sample types in coeliac disease, we demonstrated that blood samples from patients with coeliac disease on a gluten-containing diet could, for the most part, be clustered separately from blood samples from patients who do not have coeliac disease on the basis of TRB repertoires (FIG. 9).

To demonstrate the applicability of this methodology to other conditions, it was demonstrated that the method can be applied to DNA extracted from FFPE colonic biopsies and can separate biopsies from patients with Crohn's disease from those with ulcerative colitis (FIG. 12), biopsies from patients with Crohn's disease from normal (FIG. 10) and biopsies from patients with ulcerative colitis from normal (FIGS. 11a, 11b and 11c). It was also demonstrated that this method can be used to predict prognosis, for example separating melanoma cases without metastasis or recurrence (after 2 years' follow-up) from melanoma cases with both metastasis and recurrence (after 2 years' follow-up) (FIG. 14). Furthermore, it was demonstrated that clustering into three separate disease status groups, for example Crohn's disease, ulcerative colitis and normal (FIG. 13), is possible, indicating that separation into a larger number of disease status groups is possible. It was also demonstrated that the method can be applied to data produced by the analysis of RNA derived from blood samples (FIG. 18) and that the methodology is applicable to IGH, that is the B-cell receptor repertoire (FIG. 18), as well as the T-cell receptor repertoire.

In order to refine k-mer analysis further, it was demonstrated that clustering can be performed on the basis of k-mers derived from more than one locus, for example separating coeliac disease from normal on the basis of TRG and TRD, combined with a TRG:TRD=1:4 weighting (that is, using a 4:1 ratio) (FIG. 7d) and separating ulcerative colitis from normal on the basis of TRB and TRG combined with a TRB:TRG=1:1 weighting (that is, using a 1:1 ratio) (FIG. 11c). It was also demonstrated that annotating the k-mer with its position within the CDR3 sequence (for example, beginning, middle or end) can improve clustering (positionally annotated k-mers shown in FIG. 6b for comparison with use of k-mers without positional annotation in FIG. 6a; positionally annotated and unannotated k-mer use in contrastive principal component analysis is shown in FIG. 16). These results infer that k-mers may be annotated analogously with other data, including information about associated CDR1, CDR2 and CDR4/HV4 sequences.

To demonstrate the applicability of k-mer analysis to multiple dimensionality reduction and classification methodologies and combinations thereof, it was shown that, in addition to a principal component analysis and clustering approach (FIGS. 3-14), other dimensionality reduction and classification approaches can be used, such as a contrastive principal component analysis and clustering approach (FIG. 15), or principal component analysis and quadratic discriminant analysis (FIG. 16 and FIGS. 17a-17d). This demonstrates that other forms of analysis, or other combinations of dimensionality reduction and classification, are also applicable to the k-mer methodology.

1-79. (canceled)

80. A computer implemented method of determining the disease status in a subject, the method comprising the steps of:
ai) obtaining nucleotide or amino acid sequence data for the T-cell and/or B-cell receptor repertoire in a plurality of samples, wherein the samples comprise at least one reference sample obtained from a subject having a known disease status and at least one control sample obtained from a subject having a known negative disease status;
aii) identifying a data set of overlapping short sequences of nucleotides or amino acids of length k, known as k-mers, present within the nucleotide or amino acid sequence data, wherein the data set further provides information on a relative position of k-mers of amino acids or bases within the T-cell receptor or B-cell receptor amino acid or base sequence;
aiii) determining k-mer frequencies of each identified k-mer in the data set from each sample, each k-mer frequency indicating the number of times each individual k-mer sequence is present within the data set of each sample;
aiv) normalizing the k-mer frequencies to give a relative k-mer frequency, by scaling the k-mer frequencies by the total number of k-mers identified in the nucleotide or amino acid sequence data from that sample;
av) generating a matrix containing the relative k-mer frequencies of each k-mer observed in each of the samples;
avi) reducing dimensionality of the matrix to generate a reduced data set of k-mer frequencies;
avii) using a selected subset of the reduced data set of k-mer frequencies to classify the samples;
aviii) optimising one or more parameters of the data set to optimise classification of each reference sample into its known disease status, said optimised parameters comprising one or more of:
1. the set of principal components, or similarly reduced data sets of k-mer frequencies, used in the classification;
2. the length, k, of the k-mers;
3. the degree of scaling of the k-mer frequencies;
4. the relative positions of the k-mers of amino acids or bases within the T-cell receptor or B-cell receptor amino acid or base sequence, and/or whether or not they are within the CDR3 sequence; and
5. annotation of the k-mers, such that the frequencies of differentially annotated k-mers, even if they have the same amino acid or base sequence, are considered separately, such k-mer annotation comprising one or more of:
positional annotation of the k-mers indicating whether they were derived from the beginning, middle or end of the CDR3 region of the neucleotide or amino acid sequence data or at any specifically defined position within the CDR3 region; and/or
relative associations of k-mers with a particular CDR1, CDR2 or TRG CDR4/HV4 sequence in the nucleotide or amino acid sequence data; and
bi) repeating steps aii) to avi) for the plurality of samples of ai) and at least one unknown sample obtained from a subject having an unknown disease status;
bii) determining the one or more optimised parameters for the data set of the at least one unknown sample; and
c) classifying the unknown sample based on the optimised parameters to determine which reference sample or plurality of reference samples of known disease status the unknown sample is most similar to.

81. The method of claim 80 wherein the reduced data set of k-mer frequencies comprises principal components of the data set.

82. The method of claim 80 wherein reducing dimensionality of the matrix to generate a reduced data set of k-mer frequencies comprises the step of one of:
i) performing principal component analysis on the data set such that the reduced data set comprises the principal components of the data set;
ii) performing contrastive principal component analysis (CPCA) on the data set such that the principal components or the reduced data set comprises contrastive principal components of the data set;
iii) variable selection or feature selection on the data set such that the principal components are a dimensionality reduced data set;
iv) performing any suitable analysis on the data set that mediates dimensionality reduction; and
v) classification of the data in such way that a specific dimensionality reduction approach is not required, such as linear discriminant analysis, quadratic discriminant analysis, generalised discriminant analysis and canonical correlation analysis

83. The method of claim 80 wherein the step of classifying the unknown sample comprises one or more of: hierarchical cluster analysis; non-hierarchical cluster analysis; supervised cluster analysis; unsupervised cluster analysis; any other form of clustering; quadratic discriminant analysis; linear discriminant analysis; generalised discriminant analysis; nearest neighbour analysis; decision trees; support vector machines; logistic regression; neural networks; and any suitable technique for statistically mediated classification.

84. The method claim 80 wherein the step of classifying the unknown sample further includes the step of:
performing cluster analysis of the unknown sample with reference samples having a known disease status of coeliac disease and/or a known negative disease status of coeliac disease; or
performing cluster analysis of the unknown sample with reference samples having a first and second known disease status; or
performing cluster analysis of the unknown sample with reference samples having three or more known different known disease statuses; or
performing cluster analysis of the unknown sample with reference samples having two or more known physiological and/or or pathophysiological statuses.

85. The method of claim 80 wherein the disease status is the status of any condition mediated or modulated by the immune system.

86. The method of claim 80 wherein the disease status is a condition is selected from the group comprising an autoimmune condition, hypersensitivity, allergy, transplantation, transplant rejection, cancer, all forms of neoplasia, infectious diseases, and vaccination.

87. The method of claim 80 wherein the disease status is coeliac disease status; or gluten sensitivity status.

88. The method of claim 80 wherein the sample is a bodily fluid, such as one or more of: blood or a product derived from blood; lymph or a product derived from lymph; pericardial, pleural or ascitic (peritoneal) fluid(s); joint aspirate fluid; or urine; or wherein the sample is a biopsy, a fine needle aspirate sample or a buccal scrape.

89. The method of claim 80 wherein the sequence data of step a) is one or more of
i) a library of sequences representing the T-cell and/or B-cell receptor repertoire in the sample provided by the subject; or
ii) a library of DNA sequences representing the T-cell receptor repertoire in the sample provided by the subject; or
iii) a library of RNA sequences representing the T-cell receptor repertoire in the sample provided by the subject; or
iv) a library of protein sequences representing the T-cell receptor repertoire in the sample provided by the subject; or
v) a library of DNA sequences representing the B-cell receptor repertoire in the sample provided by the subject; or
vi) a library of RNA sequences representing the B-cell receptor repertoire in the sample provided by the subject; or
vii) a library of protein sequences representing the B-cell receptor repertoire in the sample provided by the subject.

90. The method of claim 80 wherein k-mers length ks of between 3 and 10 amino acids are used.

91. The method of claim 80 wherein the output of step d) is either a numerical assessment based on statistical classification, optionally including a ratio, percentage, proportion or relative likelihood; or a yes/no based on the outcome of statistical classification.

92. The method of claim 80 for use in the diagnosis of coeliac disease and/or gluten sensitivity.

93. The method of claim 80 for use in the diagnosis of Crohn's disease, by comparing data from a test sample of unknown disease status with data from known samples from Crohn's disease and normal; or for use in the diagnosis of ulcerative colitis, by comparing data from a test sample of unknown disease status with data from known samples from ulcerative colitis and normal; or for distinguishing between Crohn's disease and ulcerative colitis, optionally in order to avoid a diagnosis such as indeterminate colitis, by comparing data from a test sample of unknown disease status with data from known samples from Crohn's disease and ulcerative colitis; or for use in the determination of prognosis in melanoma patients, by comparing data from a test sample from a melanoma patient of unknown prognosis or outcome with data from melanoma patient samples with known prognosis or outcome; or for use in the diagnosis of autoimmune conditions, including but not limited to such as multiple sclerosis, pre- or early type I insulin-dependent diabetes mellitus, polymyositis, dermatomyositis, systemic lupus erythemtososus (SLE), rheumatoid arthritis, HLA-B27-associated arthritides (e.g., ankylosing spondylitis), autoimmune hepatitis, primary biliary cirrhosis and primary sclerosing cholangitis, by comparing data from a test sample of unknown disease status with data from known samples from one or more known autoimmune condition(s) with or without normal samples; or for use in the prediction of prognosis of autoimmune conditions (including but not limited to such as multiple sclerosis, pre- or early type I insulin-dependent diabetes mellitus, polymyositis, dermatomyositis, systemic lupus erythemtososus (SLE), rheumatoid arthritis, HLA-B27-associated arthritides (e.g., ankylosing spondylitis), autoimmune hepatitis, primary biliary cirrhosis and primary sclerosing cholangitis), by comparing data from a test sample of unknown disease status with data from samples with known severity or outcome in autoimmune conditionsor for use in the diagnosis of hypersensitivity conditions, by comparing data from a test sample of unknown disease status with data from known samples from a known hypersensitivity condition and normal or any other suitable comparator condition or physiological/pathophysiological status; or for use in the prediction of prognosis of hypersensitivity conditions, by comparing data from a test sample of unknown prognosis in a hypersensitivity condition with data from samples in a known hypersensitivity condition with unknown severity, outcome status or precipitating antigen with data from hypersensitivity condition samples with known severity, outcome status or precipitating antigen; or for use in the diagnosis of allergic conditions, by comparing data from a test sample of unknown disease status with data from known samples from a known allergic condition and normal or any other suitable comparator condition and/or physiological/pathophysiological status; or for use in the prediction of prognosis of allergic conditions, by comparing data from a test sample of unknown prognosis in a hypersensitivity condition with data from samples in a known allergic condition with unknown severity, outcome status or precipitating antigen with data from allergic condition samples with known severity, outcome status or precipitating antigen; or for use in the diagnosis of transplant rejection of any organ or tissue, by comparing data from a test sample of unknown disease status with data from known samples with a known rejection status and normal or any other suitable comparator condition or physiological/pathophysiological status; or for use in the prediction of prognosis or outcome in transplant rejection of any organ or tissue, by comparing data from a test sample of unknown disease status with data from samples with a known transplant rejection status, prognosis or outcome; or for use in the prediction of prognosis or outcome in cancer or any form of neoplasia, by comparing data from a test sample of unknown cancer or neoplasia outcome status with data from known cancer or neoplasia samples with a known prognosis or outcome status; or for use in the diagnosis of an infectious disease, by comparing data from a test sample of unknown infectious disease status with data from samples from individuals with a particular infectious disease status and normal and/or other infectious diseases; or for use in the prediction of prognosis or outcome of an infectious disease (of any organ or tissue), by comparing data from a test sample of unknown infectious disease prognosis or outcome status with data from samples from individuals with that particular infectious disease with known prognosis or outcome status; or for use in the prediction of prognosis or outcome of a vaccination, by comparing data from a test sample of unknown vaccination prognosis or outcome status with data from samples from individuals with particular prognosis or outcome statuses following vaccination; or for use in the determination of the specific response to one or more antigens following vaccination, by comparing data from a test sample of unknown vaccination response with data from samples from individuals with a known specific immune response to one or more specific antigens; or for use in the determination of the specific response to one or more antigens in the setting of infection, by comparing data from a sample of unknown infection status with data from samples from individuals with a known specific immune response to one or more specific antigens; or for use in the diagnosis or prediction of prognosis or outcome status of any particular physiological or pathophysiological state that is, at least in part, determined, mediated by or modulated by the immune system, by comparing data from a test sample of unknown diagnosis, prognosis or outcome status with data from samples with a known diagnosis, prognosis or outcome status; or for use in the determination of biological similarity with respect to the immune system of any group(s) of samples for the purpose of diagnosis; or for use in the determination of biological similarity with respect to the immune system of any group(s) of samples for the purpose of prediction of prognosis or outcome; or for use in the determination of physiological or pathophysiological state with respect to the immune system of any group(s) of samples for any purpose that involves comparison of the samples with samples from subjects with known physiological or pathophysiological states; or for use in the determination of biological similarity with respect to the immune system of any group(s) of samples for any purpose that involves comparison of the similarity or differences between the samples; or for use in the determination of whether there is a specific response to one or more antigens in a sample, by comparing data from that sample with data from samples from subjects with a known specific immune response to that or those specific antigen(s).

94. The method of claim 80, wherein the sequence data used in the k-mer analysis comprises sequences of any one or two or more of the following genes: TRA, TRB, TRG and TRD (considered components of the T-cell receptor repertoire) IGH, IGK and IGL (considered components of the B-cell receptor repertoire).

95. The method of claim 80 wherein the sequence data used in the k-mer analysis comprises sequences of TRA wherein the sequence data used in the k-mer analysis comprises sequences of TRB; and/or
wherein the sequence data used in the k-mer analysis comprises sequences of TRG; and/or
wherein the sequence data used in the k-mer analysis comprises sequences of TRD; and/or
wherein the sequence data used in the k-mer analysis comprises sequences of IGH; and/or
wherein the sequence data used in the k-mer analysis comprises sequences of IGK; and/or
wherein the sequence data used in the k-mer analysis comprises sequences of IGL; and/or
wherein the sequence data used in the k-mer analysis comprises sequences of TRA and TRB, TRG and/or TRD; and/or
wherein the sequence data used in the k-mer analysis comprises sequences of TRA and TRB, combined with a 1:1 weighting or any other weighting between 10,000:1 and 1:10,000; and/or
wherein the sequence data used in the k-mer analysis comprises sequences of TRG and TRD, combined with a 1:1 weighting or any other weighting between 10,000:1 and 1:10,000; and/or
wherein the sequence data used in the k-mer analysis comprises sequences of TRA and TRG, combined with a 1:1 weighting or any other weighting between 10,000:1 and 1:10,000; and/or
wherein the sequence data used in the k-mer analysis comprises sequences of TRA and TRD, combined with a 1:1 weighting or any other weighting between 10,000:1 and 1:10,000; and/or
wherein the sequence data used in the k-mer analysis comprises sequences of TRB and TRG, combined with a 1:1 weighting or any other weighting between 10,000:1 and 1:10,000; and/or
wherein the sequence data used in the k-mer analysis comprises sequences of TRB and TRD, combined with a 1:1 weighting or any other weighting between 10,000:1 and 1:10,000; and/or
wherein the sequence data used in the k-mer analysis comprises sequences of any combination of two, three or four of TRA, TRB, TRG and/or TRD, combined using a 1:1 weighting or any other relative weightings; and/or
wherein the sequence data used in the k-mer analysis comprises sequences of any two of IGH, IGK and/or IGL, combined with a 1:1 weighting or any other weighting between 10,000:1 and 1:10,000; and/or
wherein the sequence data used in the k-mer analysis comprises sequences of all three of IGH, IGK and/or IGL, combined with a 1:1 weighting or any other weighting between 10,000:1 and 1:10,000; and/or
wherein the sequence data used in the k-mer analysis comprises sequences of any combination of two or more of TRA, TRB, TRG, TRD, IGH, IGK and/or IGL, combined with a 1:1 weighting or any other relative weightings.

96. A system for classifying a sample, said system comprising a microprocessor and memory, wherein the sample comprises a T-cell receptor repertoire and/or a B-cell receptor repertoire, wherein the processor is configured to undertake the method of claim 80.

97. A k-mer dataset produced using the method of claim 80, derived from k-mer analysis of sequence data of TRA, TRB, TRG, TRD, IGH, IGK and/or IGL from samples of known disease status, known physiological status or known pathophysiological status, for use as a training or reference data set which can be used to classify new or test samples.

98. A computer readable medium storing instructions executable by one or more processors to perform operations according to the method of claim 80.
